SEQUENCE LISTING (77 sequences)

SEQ ID NO: 1 - DNA, 20 bp, synthetic construct
  source 1..20: mol_type = other DNA
ggtggggtgggggcgggccc

SEQ ID NO: 2 - DNA, 25 bp, synthetic construct
  source 1..25: mol_type = other DNA
caccgggtggggtgggggcgggccc

SEQ ID NO: 3 - DNA, 25 bp, synthetic construct
  source 1..25: mol_type = other DNA
aaacgggcccgcccccaccccaccc

SEQ ID NO: 4 - DNA, 20 bp, synthetic construct
  source 1..20: mol_type = other DNA
ccgggtctcacctccctccg

SEQ ID NO: 5 - DNA, 25 bp, synthetic construct
  source 1..25: mol_type = other DNA
caccgccgggtctcacctccctccg

SEQ ID NO: 6 - DNA, 25 bp, synthetic construct
  source 1..25: mol_type = other DNA
aaaccggagggaggtgagacccggc

SEQ ID NO: 7 - DNA, 20 bp, synthetic construct
  source 1..20: mol_type = other DNA
gcggccggagtgtgggctgc

SEQ ID NO: 8 - DNA, 25 bp, synthetic construct
  source 1..25: mol_type = other DNA
caccggcggccggagtgtgggctgc

SEQ ID NO: 9 - DNA, 25 bp, synthetic construct
  source 1..25: mol_type = other DNA
aaacgcagcccacactccggccgcc

SEQ ID NO: 10 - DNA, 20 bp, synthetic construct
  source 1..20: mol_type = other DNA
gccggcctggccggcacagg

SEQ ID NO: 11 - DNA, 25 bp, synthetic construct
  source 1..25: mol_type = other DNA
caccggccggcctggccggcacagg

SEQ ID NO: 12 - DNA, 25 bp, synthetic construct
  source 1..25: mol_type = other DNA
aaaccctgtgccggccaggccggcc

SEQ ID NO: 13 - DNA, 20 bp, synthetic construct
  source 1..20: mol_type = other DNA
aggcaccgagaagggagccg

SEQ ID NO: 14 - DNA, 25 bp, synthetic construct
  source 1..25: mol_type = other DNA
caccgaggcaccgagaagggagccg

SEQ ID NO: 15 - DNA, 25 bp, synthetic construct
  source 1..25: mol_type = other DNA
aaaccggctcccttctcggtgcctc

SEQ ID NO: 16 - DNA, 20 bp, synthetic construct
  source 1..20: mol_type = other DNA
gggaggtagcgggggcaagg

SEQ ID NO: 17 - DNA, 25 bp, synthetic construct
  source 1..25: mol_type = other DNA
caccggggaggtagcgggggcaagg

SEQ ID NO: 18 - DNA, 25 bp, synthetic construct
  source 1..25: mol_type = other DNA
aaacccttgcccccgctacctcccc

SEQ ID NO: 19 - DNA, 20 bp, synthetic construct
  source 1..20: mol_type = other DNA
ggggcaaggaggcaccgaga

SEQ ID NO: 20 - DNA, 25 bp, synthetic construct
  source 1..25: mol_type = other DNA
caccgggggcaaggaggcaccgaga

SEQ ID NO: 21 - DNA, 25 bp, synthetic construct
  source 1..25: mol_type = other DNA
aaactctcggtgcctccttgccccc

SEQ ID NO: 22 - DNA, 20 bp, synthetic construct
  source 1..20: mol_type = other DNA
cggggcacagggcggcttcg

SEQ ID NO: 23 - DNA, 25 bp, synthetic construct
  source 1..25: mol_type = other DNA
caccgcggggcacagggcggcttcg

SEQ ID NO: 24 - DNA, 25 bp, synthetic construct
  source 1..25: mol_type = other DNA
aaaccgaagccgccctgtgccccgc

SEQ ID NO: 25 - DNA, 20 bp, synthetic construct
  source 1..20: mol_type = other DNA
gtattactgatattggtggg

SEQ ID NO: 26 - DNA, 25 bp, synthetic construct
  source 1..25: mol_type = other DNA
caccggtattactgatattggtggg

SEQ ID NO: 27 - DNA, 25 bp, synthetic construct
  source 1..25: mol_type = other DNA
aaaccccaccaatatcagtaatacc

SEQ ID NO: 28 - DNA, 20 bp, synthetic construct
  source 1..20: mol_type = other DNA
gtcggccgagacattgcgca

SEQ ID NO: 29 - DNA, 25 bp, synthetic construct
  source 1..25: mol_type = other DNA
caccggtcggccgagacattgcgca

SEQ ID NO: 30 - DNA, 25 bp, synthetic construct
  source 1..25: mol_type = other DNA
aaactgcgcaatgtctcggccgacc

SEQ ID NO: 31 - DNA, 20 bp, synthetic construct
  source 1..20: mol_type = other DNA
cgccaagtacggcatcgagg

SEQ ID NO: 32 - DNA, 25 bp, synthetic construct
  source 1..25: mol_type = other DNA
caccgcgccaagtacggcatcgagg

SEQ ID NO: 33 - DNA, 25 bp, synthetic construct
  source 1..25: mol_type = other DNA
aaaccctcgatgccgtacttggcgc

SEQ ID NO: 34 - DNA, 20 bp, synthetic construct
  source 1..20: mol_type = other DNA
gagcgccaagtacggcatcg

SEQ ID NO: 35 - DNA, 25 bp, synthetic construct
  source 1..25: mol_type = other DNA
caccggagcgccaagtacggcatcg

SEQ ID NO: 36 - DNA, 25 bp, synthetic construct
  source 1..25: mol_type = other DNA
aaaccgatgccgtacttggcgctcc

SEQ ID NO: 37 - DNA, 15 bp, synthetic construct
  source 1..15: mol_type = other DNA
cttttgcgtcgccag

SEQ ID NO: 38 - DNA, 20 bp, synthetic construct
  source 1..20: mol_type = other DNA
ttgatggcaacaatatccac

SEQ ID NO: 39 - DNA, 20 bp, synthetic construct
  source 1..20: mol_type = other DNA
caagagccagaagtatgatg

SEQ ID NO: 40 - DNA, 20 bp, synthetic construct
  source 1..20: mol_type = other DNA
gtgaagcagtttgctttttc

SEQ ID NO: 41 - DNA, 20 bp, synthetic construct
  source 1..20: mol_type = other DNA
gtgtcctcacagcgtacctc

SEQ ID NO: 42 - DNA, 20 bp, synthetic construct
  source 1..20: mol_type = other DNA
ccgttactccagagacccct

SEQ ID NO: 43 - DNA, 23 bp, synthetic construct
  source 1..23: mol_type = other DNA
ggtgaaacccaaacatagtctcg

SEQ ID NO: 44 - DNA, 22 bp, synthetic construct
  source 1..22: mol_type = other DNA
ttgtgcaagtgcaagattctca

SEQ ID NO: 45 - DNA, 20 bp, synthetic construct
  source 1..20: mol_type = other DNA
cattcgcttagttggtctac

SEQ ID NO: 46 - DNA, 20 bp, synthetic construct
  source 1..20: mol_type = other DNA
ttctaccgtttttcagcttc

SEQ ID NO: 47 - DNA, 36 bp, synthetic construct
  source 1..36: mol_type = other DNA
taagcaggtaccacaggacactcctgtgctcccatg

SEQ ID NO: 48 - DNA, 35 bp, synthetic construct
  source 1..35: mol_type = other DNA
tgcttactcgagctttagtggagccaccctccagg

SEQ ID NO: 49 - DNA, 36 bp, synthetic construct
  source 1..36: mol_type = other DNA
taagcaggtaccactgctaaggacaagggtctctcg

SEQ ID NO: 50 - skipped

SEQ ID NO: 51 - DNA, 36 bp, synthetic construct
  source 1..36: mol_type = other DNA
taagcaggtaccccctggcctccatcattgctaagg

SEQ ID NO: 52 - DNA, 35 bp, synthetic construct; identical to SEQ ID NO: 48
  source 1..35: mol_type = other DNA

SEQ ID NO: 53 - DNA, 36 bp, synthetic construct; identical to SEQ ID NO: 47
  source 1..36: mol_type = other DNA

SEQ ID NO: 54 - DNA, 36 bp, synthetic construct
  source 1..36: mol_type = other DNA
tgcttactcgagcgagagacccttgtccttagcagt

SEQ ID NO: 55 - DNA, 36 bp, synthetic construct; identical to SEQ ID NO: 49
  source 1..36: mol_type = other DNA

SEQ ID NO: 56 - DNA, 36 bp, synthetic construct
  source 1..36: mol_type = other DNA
tgcttactcgagccttagcaatgatggaggccaggg

SEQ ID NO: 57 - DNA, 36 bp, synthetic construct; identical to SEQ ID NO: 51
  source 1..36: mol_type = other DNA

SEQ ID NO: 58 - DNA, 35 bp, synthetic construct; identical to SEQ ID NO: 48
  source 1..35: mol_type = other DNA

SEQ ID NO: 59 - DNA, 34 bp, synthetic construct
  source 1..34: mol_type = other DNA
taagcaggtacccagtcccaactcgtacctcgca

SEQ ID NO: 60 - DNA, 36 bp, synthetic construct
  source 1..36: mol_type = other DNA
tgcttactcgagctcaccagtcttctctcctggcag

SEQ ID NO: 61 - DNA, 20 bp, synthetic construct
  source 1..20: mol_type = other DNA
gcgactatacgcgcaatatg

SEQ ID NO: 62 - DNA, 20 bp, synthetic construct
  source 1..20: mol_type = other DNA
tgtgagaacacccccttcag

SEQ ID NO: 63 - DNA, 20 bp, synthetic construct
  source 1..20: mol_type = other DNA
ctccacctgtacccctttga

SEQ ID NO: 64 - DNA, 20 bp, synthetic construct
  source 1..20: mol_type = other DNA
gcttcccatgtatgcttcaa

SEQ ID NO: 65 - DNA, 20 bp, synthetic construct
  source 1..20: mol_type = other DNA
tttgggtttcaccagttctt

SEQ ID NO: 66 - skipped

SEQ ID NO: 67 - DNA, 20 bp, synthetic construct
  source 1..20: mol_type = other DNA
tctggcgtctcgcagatgta

SEQ ID NO: 68 - DNA, 20 bp, synthetic construct
  source 1..20: mol_type = other DNA
ttgtccccgtccagtttgag

SEQ ID NO: 69 - DNA, 18 bp, synthetic construct
  source 1..18: mol_type = other DNA
ggtctcacctccctccga

SEQ ID NO: 70 - DNA, 22 bp, synthetic construct
  source 1..22: mol_type = other DNA
tcactcaaagccgctcaactac

SEQ ID NO: 71 - DNA, 24 bp, synthetic construct
  source 1..24: mol_type = other DNA
tctgccttcatttcgttatgtacc

SEQ ID NO: 72 - RNA, 2884 nt, Homo sapiens
  source 1..2884: mol_type = transcribed RNA
gcagatgtacaccgcgtcgtcgtcggccgagacattgcgcacggtcaggcggcgctcggtgccctcctcctcgatgccgtacttggcgctcgcccacaaccgcgtctcctccttgaaccacgcggcctcagtggacggctggggcacctcacataggaacgtggccgactccttctcccgcacctccagatcttgcagccgctttttgaagggaaccgcgggctctgccaggagagaagactggaacggacacctgcaaccctggattccacgaccggacagacgcgggtgggaaacggcagaccgcaagggcgaaaggacccaggtgcacgggttgcggaggagcctgggcccgcgcgccccgcacccaggagctcaccgcgcactacgaccagcacagagctgtaggtctggcccacgaggttggacgccgtgcaggtgtagaggccgcggtccgactgcttgcagaagaggatcttgagcacgaagttctcctgcgcgtcctcgtacaccacgtggcgccggccctcggtcaccagctggccgtccttcttccacaccgtctcgggcttgggctcgccggtcacgtagcagctgaggcgcgcgtgcttgccttcagtcaccgtgcaggtgcctgctgtcccagctactcaagagactgaagcaggaggatggcttgagcccaggaggtcaaagctgcagtgagctgcttcgccccatccctgatccagtgccaggcctctatgtgtcctcacagcgtacctcaggctcttccgggcaatggccaacatctctcatgtgtcctcaatcagaaggagggtgaggggtctctggagtaacggttatgccattctgcagccttatctcatgactgatcacttccaaaagctcacgttctaatcctgtcaccttgggggtgagcatttcaacgtgtgaatctccgggacacacattcagtccacagcaccagggagaaaggaaaggctggaaggagagcccactcccccaccaggagcttggccctgagctgccccagccatgcagaagtgtgtgtgcattgtgtgtgctcgaatacgtgccccaccccctcccggacagggccgtggagggtgcagagaagatggctgtgcgggccagccccttgcagggcctcctggacctgggagaagcaatgcccccacagcctccaggagtggcctgccctcctgaccctcttcactgctgcctgctccgctgcatctccaaacaccactgccagccttcaggctggattttctgcctccttcacaggacccctgttgttccttatttccctgcaacgtatttgttgaagacagtgggatattcacactacagagcttccggaggcactgtgcccagggtgtccttcacaccacgggaggccacaggatgcagtggctggccatggaccttgcctattcctggctggaatccactgcaaagagaactttcatggagctgctgtctgttcaaaggggtacaggtggaggaggagcagacacaatttcctgaggaaagcaaatgtcactgtgtcccttgagttgccagcttccacagggatgtcccgccatgagccctcctcccaggggctttcacagctgggtgggctggcatcctgggtgcagtttaactgaagggggtgttctcacagccatctcccaaggcctgggggatggtgggcccggaggcctcagacctgcacctgctgcccaggcgatggggtggctgggccccgggtgcccacaaactataggcaaaaaaaactgtaagtcagtcctgcagggtttcctgaacattttatgatccccaagtcgtatctgaacatgtcaagaatctctggtgccttggcttgggtgtaaagatgaagttctttcctcctctgtattccaggaggaaagagggttttcattcccttctgaatgttaggagggaaggcattttccatccttgaccagcttgtagatgacacacattcagcaaacgcacatcccagcagcaccgagagaacacacaggtgtgtcacgcttttgcgttcataaggccttttcatgtcagtatcttattagatattcccaaacaagggtgttttctcatcttatagaagagaggcctgaggctcacagggtgaaaacaatttgcttaagcctgcacagcagctgtgctggcaacgtttctcccggtataaatgagaagcagcctttgtggccacagagatgtgactgctaagtgcatcgtcccaccctgaaggagattcttcctcaacaagaggacattagtgcaaagactgatgaagtctgtagtttcgtgggttttttttttaagcataaggggttaaatattatgtaattaagtaataaggcagttgtgaggatgcaactgccaaaagtttgaaaaaagctgcccagatggaaatctgcttgcatttgtgcaagcagataggtgagggatttttactgtactgccgtgtcccctacctgccccagctttattgagttataattgacaagcaaaaattgtttatatttaaagtgtacaatgtgatgtggcctttttgttttgttttgagacataatcttatcacccaggctggagtgaagtggcgcagtttaaactgcgcaggctcatcatcctccttcctcagcctcctcagtagctagaaccacaggtgtgagccaccatgcctggctcattttttgattttctttttttaagagacaggggtctccctgtttcccaggctggtcttgaactcccgggctcaagtaatcatcccgcctcagcctcccaaagtgatggtgttacaggcatgagctactgtgcctggccagaaatggtaaattataattaaaggcttctgtaagtc

SEQ ID NO: 73 - RNA, 981 nt, Homo sapiens
  source 1..981: mol_type = transcribed RNA
caccgcgtcgtcgtcggccgagacattgcgcacggtcaggcggcgctcggtgccctcctcctcgatgccgtacttggcgctcgcccacaaccgcgtctcctccttgaaccacgcggcctcagtggacggctggggcacctcacataggaacgtggccgactccttctcccgcacctccagatcttgcagccgctttttgaagggaaccgcgggctctgccaggagagaagactgctattttgaagcatacatgggaagctggttcctggacccccgcatataccaaaatctgtgcatactcaagttctacggtcagccctgtggaacccaagcataggacaagccggccctccctatacacacaggtttgacatcctgagaacactgctggttcccttcccatttggttccagaaaatctgcgtataagtggactcagtggtgcaaacctatgttgttcaagggtcaactatatatgatgaattattgttacctacagtcactctaccatgctactaaacactagaactgattcattttctctgactgtgtgtctctacccattaaccagcccctcttcatctcccttcccgcttccctccccagcctctggtaaccatcattcagttctctacctccgtgaaatcaacatgagtaagaactggtgaaacccaaacatagtctcgaatttagctttttttttttttttaactgtaagaggctaaacattcttgaggaggacacaaaaaagtctgaagacaactgtccagatgagaatcttgcacttgcacaagtagagaggagagttagtttttataccatcgcatccccaatcccagctttattgatgcataacaaataaaaattgtacatatttaaagtgtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 74 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 75 - protein, 50 aa, synthetic construct
DALDDFDLDMLGSDALDDFDLDMLGSDALDDFDLDMLGSDALDDFDLDML

SEQ ID NO: 76 - DNA, 19 bp, synthetic construct
  source 1..19: mol_type = other DNA
acatgaggatcacccatgt

SEQ ID NO: 77 - DNA, 13500 bp, synthetic construct
  source 1..13500: mol_type = other DNA
ggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtacctgaggtgtgactggaaaacccacctcctcctcctcttgtgcttctagccaggcacaatcagcattggtagctgctgtattgctacttgtgattgctccatgtttttctaggtctcgaggtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattaccgataagcttgatatcgaattcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccattgggccaggattctcctcgacgtcaccgcatgttagcagacttcctctgccctctccactgcctgtacagttaatcagcatgtccaggtcgaaatcatcaagggcgtcactgccgagcatgtcgaggtcaaagtcatcaagggcatccgaaccaagcatgtcaaggtcaaaatcatcgagggcgtcacttcccagcatatccagatcaaaatcgtccaatgcgtcagcccgtccggatccagcggccgccaccttcctctttttcttaggtccgctacctcctcctccgcttcctcctcctccgcttccacctcctccagcgctgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccgggccttgtcgcttctggtcagcaccttgttgtcgatggagtcgtccttcagaaagctctgaggcacgatgtggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggttgccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatggccaggccgatgctgtacttcttgtcctttttcttttttgcctggccggcctttttcgtggccgccggccttttcatggtggccgtacgcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagccaattcccactcctttcaagacctaggatccaaaaaaagcaccgactcggtgccacttggccctgcagacatgggtgatcctcatgttggccaagttgataacggactagccttattttaacttgctaggccctgcagacatgggtgatcctcatgttggcctagctctaaaacagagacggtatcccgtctccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtc

(19) United States
(12) Patent Application Publication
(10) Pub. No.: US 2025/0339556 A1
(43) Pub. Date: Nov. 6, 2025

(54) COMPOSITIONS AND METHODS FOR TREATING CANCER BY INCREASING EXPRESSION OF OBSCN-AS1 LONG-NONCODING RNA

(71) Applicants: University of Maryland, Baltimore, Baltimore, MD (US); The Johns Hopkins University, Baltimore, MD (US)

(72) Inventors: Aikaterini Kontrogianni-Konstantopoulos, Ellicott City, MD (US); Talia Guardia, Frederick, MD (US); Konstantinos Konstantopoulos, Ellicott City, MD (US)

(21) Appl. No.: 18/854,792

(22) PCT Filed: Apr. 5, 2023
(86) PCT No.: PCT/US2023/065366

§ 371 Date: Oct. 7, 2024

Related U.S. Application Data
(60) Provisional application No. 63/327,485, filed on Apr. 5, 2022.

(51) Int. Cl. A61K 48/00; A61K 38/17; A61K 45/06; A61P 35/00; C12N 9/22; C12N 15/11; C12N 15/86; G01N 33/50

(52) U.S. Cl. CPC A61K 48/005; A61K 38/1709; A61K 45/06; A61P 35/00; C12N 9/226; C12N 15/111; C12N 15/86; G01N 33/5023; G01N 33/5044; C07K 2319/71; C12N 2740/15043; G01N 2333/4703; G01N 2800/52

(57) ABSTRACT

The present disclosure unravels novel mechanistic information involving the direct regulation of OBSCN via OBSCN-AS1 through chromatin remodeling and enhanced RNA polymerase II recruitment. Remarkably, it is shown herein that targeting of OBSCN-AS1 is sufficient to restore OBSCN expression in highly aggressive triple-negative breast cancer cells. Provided herein are methods for increasing OBSCN expression in a cell, comprising providing to the cell one or more agents that increases levels of OBSCN-AS1 IncRNA or a variant thereof in the cell, and CRISPR/Cas9 systems for increasing levels of OBSCN-AS1 IncRNA in cells.

### CROSS-REFERENCE TO RELATED APPLICATIONS

This application claims the benefit of U.S. Provisional Appl. No. 63/327,485, filed on Apr. 5, 2022, the contents of which are hereby incorporated by reference in their entirety.

### STATEMENT OF FEDERALLY SPONSORED RESEARCH AND DEVELOPMENT

This invention was made with government support under the Grant Number CA183804 awarded by the National Institutes of Health. The government has certain rights in the invention.

### FIELD OF THE INVENTION

The field of the invention relates to cancer, in particular compositions and methods for treating breast cancer.

### BACKGROUND OF THE INVENTION

Breast cancer remains the second leading cause of cancer death among women with 1 in 8 women predicted to develop invasive breast cancer over the course of her lifetime in the U.S. (breastcancer.org (U.S. Breast Cancer Statistics. (breastcancer.org)). Despite death rates decreasing by 1% per year from 2013 to 2018 owing to increased awareness, early detection, and treatment advancements, an estimated 287,850 and 51,400 new cases of invasive and non-invasive (in situ) breast cancer are expected to be diagnosed in 2022 (breastcancer.org (U.S. Breast Cancer Statistics. (breastcancer.org)). Sadly, 43,250 women are predicted to succumb to the disease (breastcancer.org (U.S. Breast Cancer Statistics. (breastcancer.org)), as once it progresses to metastatic disease survival drops sharply. Accordingly, recent statistics indicate that ˜99% of women diagnosed with localized breast cancer will survive 5 years in comparison to ˜28% with metastatic disease (American Cancer Society (Survival Rates for Breast Cancer)). Thus, metastasis, treatment resistance, and recurrence have remained major challenges, underscoring the unmet need for developing novel predictive, diagnostic, and therapeutic targets for metastatic breast cancer.

Extensive research has scrutinized the role, alterations, and signaling interplay of protein-coding genes in breast cancer initiation and progression. However, recently the role of noncoding genes giving rise to long noncoding RNAs (lncRNAs) as key regulators of breast tumorigenesis and metastasis, potent biomarkers, and modulators of drug resistance and sensitivity has been highlighted (Liu et al., Mol Cancer, (2020), 19(54)). Similar to mRNAs, lncRNAs are transcribed by RNA polymerase II and can undergo alternative splicing (Fernandes et al., Noncoding RNA, (2019). 5). Their typical length is ≥200 nucleotides (nts) and depending on their orientation and position in the genome they are classified as intergenic, intronic, bidirectional, enhancer, and antisense lncRNAs (Fernandes et al., Noncoding RNA, (2019), 5). Antisense lncRNAs are transcribed from the complementary strand of coding or non-coding genes with which they may partially or entirely overlap (Fernandes et al., Noncoding RNA, (2019), 5). Strand-specific transcriptomic studies using breast cancer biopsies have indicated the concordant expression of non-coding lncRNA/protein-coding gene pairs, suggesting their functional interplay (Balbin et al., Genome Res 25, (2015), 1068-1079; Wenric et al., Sci Rep 7, (2017), 17452). Accordingly, lncRNAs have been shown to play essential roles in diverse cellular processes, including cell cycle control (7), transcription and translation via cis- or trans-factor recruitment (8), and epigenetic regulation including both DNA methylation and histone modification (9) of their protein-coding partners (Kitagawa et al., Cell Mol Life Sci 70, (2013), 4785-4794; Long et al., Sci Adv 3, (2017), eaao2110; Angrand et al., Front Genet 6, (2015), 165; Vance et al., Trends Genet 30, (2014), 348-355).

OBSCN-Antisense RNA 1 (OBSCN-AS1) is a lncRNA gene located in human chromosome 1q42.13 that originates from the minus strand of the protein-coding OBSCN gene (Guardia et al., Biochim Biophys Acta Rev Cancer 1876, (2021) 188567). Two splice variants of OBSCN-AS1 have been described with variant-1 (2884 nts) consisting of 4 exons and variant-2 (981 nts) containing 2 exons. As the molecular identity of OBSCN-AS1 was recently unraveled, its functional significance has yet to be elucidated. Conversely, mounting evidence has implicated OBSCN, encoding the giant cytoskeletal proteins obscurins (720-870 kDa), in the predisposition and development of different cancer types (Guardia et al., Biochim Biophys Acta Rev Cancer 1876, (2021) 188567). Earlier work identified OBSCN and TP53 as two commonly mutated genes in breast and colorectal cancers, while recent bioinformatics studies identified OBSCN as a candidate driver gene in breast tumorigenesis that exhibits ˜18% average alteration frequency according to cBioPortal datasets (Sjoblom et al., Science 314, (2006), 268-274; Rajendran et al., Oncotarget 8, (2017), 102263-102276; Rajendran et al., Oncotarget 8, (2017), 50252-50272). In agreement with these observations, evaluation of the mutational frequency of OBSCN across 33 different cancer types using the TCGA PanCancer Atlas indicated that it ranges from ˜0.6% in well-differentiated thyroid cancer to >30% in undifferentiated stomach adenocarcinoma, with an estimated ˜12.5% in breast cancer (Guardia et al., Biochim Biophys Acta Rev Cancer 1876, (2021) 188567). More importantly, OBSCN expression is markedly reduced in advanced stage breast cancer biopsies (Shriver et al., Oncogene 34, (2015), 4248-4259). Consistent with this observation, the sole depletion of OBSCN from breast epithelial cells promotes survival, anoikis evasion and chemoresistance, induces epithelial-to-mesenchymal transition (EMT) and stemness, and increases their migratory, invasive, and re-attachment potentials (Shriver et al., Oncogene 34, (2015), 4248-4259; Perry et al., FASEB J 26, (2012) 2764-2775). Dysregulation of the RhoA and PI3K/Akt signaling axes was found to be downstream of OBSCN loss, both of which are frequently altered in invasive breast carcinomas (Perry et al., Oncotarget 5, (2014), 8558-8568; Tuntithavornwat et al., Cancer Lett 526, (2022), 155-167; Shriver et al., Oncotarget 7, (2016), 45414-45428; Miricescu et al., Int J Mol Sci 22, (2020); Humphries et al., Cells 9 (2020)).

Despite the advancement of knowledge regarding the pivotal role of OBSCN in breast tumorigenesis and metastasis, its regulation in healthy breast epithelium and how it is altered in breast cancer cells has remained largely elusive.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

The present disclosure unravels novel mechanistic information involving the direct regulation of OBSCN via OBSCN-AS1 through chromatin remodeling and enhanced RNA polymerase II recruitment. Remarkably, it is shown herein that targeting of OBSCN-AS1 is sufficient to restore OBSCN expression in highly aggressive triple-negative breast cancer (TNBC) cells, drastically suppressing their migratory, invasive, and metastatic potential in vitro and in vivo. Collectively, these findings demonstrate that OBSCN-AS1 functions upstream of OBSCN to regulate its transcriptional activation and implicate the OBSCN-AS1/OBSCN gene pair as potent metastasis suppressors in breast cancer and prominent therapeutic targets.

In one aspect, the invention provides a method for increasing OBSCN expression in a cell, comprising providing to the cell one or more agents that increases levels of OBSCN-AS1 lncRNA or a variant thereof in the cell.

In another aspect, the invention provides a method of treating cancer in a subject, comprising administering to the subject an effective amount of one or more agents that increases levels of OBSCN-AS1 lncRNA or a variant thereof in cancer cells of the subject.

In another aspect, the invention provides a CRISPR/Cas system for increasing OBSCN expression in a cell, comprising i) a nucleic acid encoding a sgRNA comprising a targeting domain which is complementary with a target sequence of the OBSCN-AS1 gene and ii) a nucleic acid encoding a Cas9 polypeptide or a variant thereof.

In another aspect, the invention provides a method of prognosing cancer in a subject, comprising
i) providing cancer cells from the subject;
ii) assaying the cells for expression of OBSCN and comparing OBSCN expression level to a control; and
iii) assaying the cells for expression of OBSCN-AS1 and comparing OBSCN-AS1 expression level to a control;
   wherein reduced expression level of OBSCN and/or OBSCN-AS1 relative to the control indicate an increased probability for metastasis,
   wherein normal or increased expression level of OBSCN and/or OBSCN-AS1 relative to the control indicate an increased sensitivity to an anthracycline chemotherapeutic agent.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. OBSCN-AS1 is a nuclear lncRNA that, like its protein coding partner OBSCN, shows reduced expression in human breast cancer biopsies and cell lines. (A) Schematic representation of the OBSCN-AS1/OBSCN gene pair genomic loci. Figure adapted from Guardia et al. (2021). The OBSCN (chr1: 228,208,044-228,378,876), GUK1 (chr1: 228,140,084-228,148,955), and IBA57 (chr1: 228,165,804-228,182,257) genes are encoded by the (+) strand while the OBSCN-AS1 (chr1: 228,203,506-228,213,664) and TRIM11 (chr1: 228,393,672-228,406,835) genes are encoded by the (−) strand; the coordinates provided are based on the GRCh38.p13 assembly release. (B) OBSCN gene expression is significantly reduced in breast invasive carcinoma tumor samples (n=112) compared to normal adjacent tissue (n=112) as shown from analysis of RNA-seq data using the TNM plot web tool (tnmplot.com; p-value=6.1e-13, Mann-Whitney test). (C) Similarly, OBSCN-AS1 gene expression is markedly decreased in breast invasive carcinoma tumor samples (n=112) compared to normal adjacent tissue (n=112; p-value=2.72e-04, Mann-Whitney test). (D) Analysis of the OBSCN mRNA and OBSCN-AS1 lncRNA expression profiles in breast invasive carcinoma tumor samples using the TANRIC platform showed a positive correlation (https://www.tanric.org; spearman correlation=0.7363494, p-value=0). (E-F) The expression levels of OBSCN and OBSCN-AS1 are significantly reduced in TNBC (n=164) compared to normal (n=32) tissue samples, as shown from analysis using publicly available transcriptome microarray data (GSE76250). Data are represented as mean±SEM of n=3 independent experiments; **p<0.01 and ***p<0.001 using two-tailed t-test. (G) Analysis of OBSCN and OBSCN-AS1 expression in TNBC samples revealed that they exhibit positively correlated expression values. (H-I) RT-qPCR evaluation of the OBSCN-AS1 lncRNA variant 1 and 2 transcripts (H) and the OBSCN mRNA (I) levels indicated that they are significantly lower in the TNBC cell lines MDA-MB-231 and Hs578T compared to the non-tumorigenic MCF10A breast epithelial cells. Data are represented as mean±SD of n=3 independent experiments; ***p<0.001 and ****p<0.0001 using one-way ANOVA with Dunnett's multiple comparisons. (J) Kaplan-Meier plot indicated that breast cancer patients with low OBSCN-expressing tumors exhibit significantly reduced overall survival compared to patients with high OBSCN-expressing tumors; the analysis was performed by Betastasis (betastasis.com) using the TCGA BRCA dataset (median preset expression threshold, logrank p-value=0.00366). (K) Similarly, Kaplan-Meier analysis of the relapse-free survival (RFS) probability of breast cancer patients demonstrated that patients with low OBSCN-expressing tumors exhibit markedly reduced RFS compared to patients with high OBSCN-expressing tumors; the analysis was performed by Kaplan-Meier Plotter (kmplot.com; Hazard Ratio (HR)=0.72, logrank p-value=2.1e-05, FDR: 2%). (L) RT-qPCR analysis using cytoplasmic and nuclear RNA fractionated from MCF10A cells demonstrated that OBSCN-AS1 variants 1 and 2 transcripts are exclusively found in the nucleus; data are represented as mean±SD of n=3 independent experiments.

FIG. 2. Identification of the OBSCN and OBSCN-AS1 promoter in breast epithelial cells. (A) Schematic representation of the luciferase constructs containing successive or overlapping regions of the OBSCN promoter; the coordinates provided are based on the GRCh38.p13 assembly release. (B) Dual luciferase reporter assays of the OBSCN promoter regions 1-5 following transient transfection in MCF10A cells; luciferase constructs containing empty vector and the TK promoter were used as negative and positive control, respectively. Region 3 containing −235 bp to +205 bp from the TSS elicited the highest luciferase transcriptional activity. (C) Schematic representation of the luciferase constructs containing the OBSCN (i.e., region 3) and OBSCN-AS1 promoters as identified in our experimental set-up; the coordinates provided are based on the GRCh38.p13 assembly release. (D) Dual luciferase reporter assays of OBSCN (i.e., region 3) and OBSCN-AS1 promoter constructs following transient transfection in MCF10A cells demonstrated their ability to induce luciferase activity; as before, luciferase constructs containing empty vector and the TK promoter were used as negative and positive control, respectively. Data are represented as mean±SD of n=3 independent experiments; in panels B and D, for comparisons of OBSCN and OBSCN-AS1 luciferase constructs versus empty vector negative control *p<0.05, **p<0.01, and ***p<0.001 using one-way ANOVA with Dunnett's multiple comparisons test; in panel D comparison of OBSCN versus OBSCN-AS1 promoter luciferase constructs *p<0.05 using two-tailed t-test.

FIG. 3. CRISPR-activation of the OBSCN-AS1 promoter leads to robust induction of OBSCN-AS1 and OBSCN expression. (A) Schematic illustration of the genomic location of the sgAS1-4 RNAs targeting the OBSCN-AS1 promoter (region shown chr1:228,213,595-228,213,825); sgAS1-4 target sequences partially overlap and are shown as color-shaded areas with sgAS1 in blue, sgAS2 in red, sgAS3 in green, and sgAS4 in pink. (B-C) RT-qPCR analysis of MDA-MB-231 (B) and Hs578T (C) cells transduced with sgAS1-4 lentiviruses demonstrated significant or trending upregulation of OBSCN-AS1 lncRNA variant 1 and 2 expression with select constructs (i.e., sgAS2 and sgAS3). (D-E) Similarly, RT-qPCR analysis showed marked increase of OBSCN mRNA levels, too, in both MDA-MB-231 (D) and Hs578T (E) cells transduced with sgAS2 and sgAS3. (F-G) The robust upregulation of OBSCN transcripts was followed by significant increase of obscurin protein levels in sgAS2 and sgAS3 expressing MDA-MB-231 cells (F) and sgAS2 transduced Hs578T cells (G), as shown by densitometric evaluation of the relevant immunoblots. Data are represented as mean±SD of n>3 independent experiments; *p<0.05, **p<0.01, ***p<0.001 and ****p<0.0001 using one-way ANOVA with Dunnett's multiple comparisons (B, C and F), Kruskal-Wallis test with Dunn's multiple comparisons (D and E), and Kruskal-Wallis test uncorrected with Dunn's multiple comparisons (G).

FIG. 4. OBSCN-AS1 regulates OBSCN transcription through chromatin remodeling. (A) Visual representation of the antisense oligonucleotides ASO1-4 target-regions in OBSCN-AS1 transcript variants 1 and 2. ASO 1-4 target regions are shown as color-shaded areas with ASO-1 in blue, ASO-2 in red, ASO-3 in green, and ASO-4 in pink; NC is a non-targeting control ASO. (B) Antisense oligonucleotide (ASO)-mediated knockdown of OBSCN-AS1 in sgAS2 transduced MDA-MB-231 cells followed by RT-qPCR analysis indicated significant downregulation of both variants 1 and 2, which was more pronounced in cells simultaneously treated with ASO-1 and ASO-4. (C) RT-qPCR analysis further showed significant reduction of OBSCN mRNA expression in sgAS2 transduced MDA-MB-231 cells treated with ASO-1 or ASO-4, that was most prominent when ASO-1 and ASO-4 were used in combination. (D-E) Similarly, RT-qPCR analysis of MCF10A cells simultaneously treated with ASO-1 and ASO-4 indicated significant knockdown of OBSCN-AS1 variants 1 and 2 (D) that was accompanied by marked downregulation of OBSCN mRNA expression, too (E). (F-G) ChIP-qPCR analysis demonstrated significantly increased binding of RpbI (the largest subunit of RNA polymerase II) to both the OBSCN-AS1 and OBSCN promoter (F) and marked enrichment of H3K4me3 levels at the OBSCN promoter locus (G) in sgAS2 transduced MDA-MB-231 cells compared to EV controls. (H) RT-qPCR analysis of MCF10A CRISPRi (dCas9-KRAB) cells transduced with sgAS38, sgAS71, and sgAS74 lentiviruses demonstrated significant knockdown of OBSCN-AS1 lncRNA variant 1 and 2. (I) Similarly, RT-qPCR analysis showed markedly reduced OBSCN mRNA levels. (J-L) ChIP-qPCR analysis demonstrated significantly decreased RpbI binding (J) and H3K4me3 levels (K) but marked enrichment of H3K9me3 levels (L) at both the OBSCN-AS1 and OBSCN promoter in sgAS38 transduced MCF10A cells compared to sgCtrl cells. Data are represented as mean±SD of n>3 independent experiments; *p<0.05, **p<0.01, ***p<0.001 and ****p<0.0001 calculated using one-way ANOVA with Dunnett's multiple comparisons (panels B-C: individual use of ASOs 1-4 compared to NC, and panels H-I), and two-tailed t-test (panels B-C: combined use of ASOs 1 and 4 compared to NC, and panels D, E, F, G, J, K and L).

FIG. 5. OBSCN-AS1/OBSCN upregulation in MDA-MB-231 cells reduces cell migration but does not alter cell proliferation. (A) MDA-MB-231 cells transduced with sgAS2 and sgAS3 show significantly reduced wound closure compared to EV control cells 9 h post-wound in wound healing assays. (B) Transwell migration of sgAS2 and sgAS3 expressing MDA-MB-231 cells is markedly decreased compared to EV control cells. (C) Representative images of MDA-MB-231 EV control, sgAS2, and sgAS3 transduced cells migrating through microchannels (10 μm in height and 3 μm in width) in a microfluidic device; yellow arrows point to the position of individual cells within microchannels. (D-I) Single cell migration analysis demonstrated that MDA-MB-231 cells expressing sgAS2 and sgAS3 exhibit significantly reduced percent cell entry (D), increased cell entry time (E), unaltered longitudinal area (F), and decreased velocity (G), speed (H), and persistence (I) compared to EV control cells. See also Supplemental video 1. (J) Cell proliferation of MDA-MB-231 cells expressing EV, sgAS2 or sgAS3 was unaffected, as determined by the number of living cells during a 96 h period. Data in (A-B) are represented as mean±SD of n=3 independent experiments; **p<0.01 using one-way ANOVA with Dunnett's multiple comparisons; data in (D-I) are represented as mean±SD of n>28 cells (D, E, and G-I) or n=52 cells (F) from 3 independent experiments; NS=non-significant and ***p<0.001 using one-way ANOVA with Tukey's multiple comparisons test (D, F) or Kruskal-Wallis test with Dunn's multiple comparisons test (E, and G-I); and data in (J) are represented as mean±SD of n=3 independent experiments; NS=non-significant using two-way ANOVA with Dunnett's multiple comparisons test.

FIG. 6. OBSCN-AS1/OBSCN upregulation in MDA-MB-231 cells reduces collective cell migration and invasion in a 3D spheroid model. (A) Representative images of spheroids generated from EV, sgAS2 and sgAS3 expressing MDA-MB-231 cells embedded in 3D-collagen gels; images were taken immediately at t=0 h and t=24 h following spheroid transfer into 3D collagen. Yellow arrows denote cell protrusions from spheroids generated from EV, but not sgAS2 or sgAS3, MDA-MB-231 cells, indicative of cell migration and invasion into the collagen matrix. (B-C) Initial assessment at t=0 h indicated that spheroids generated from MDA-MB-231 sgAS2 and sgAS3 cells display significantly increased spheroid area (B) and circularity (C) compared to EV controls. (D-G) Cell migration and invasion measurements over a period of 24 h demonstrated that sgAS2 and sgAS3 transduced MDA-MB-231 cells exhibit significantly increased first cell dissociation time (D), diminished normalized area expansion (E), decreased mean square displacement (MSD) and trajectory (F), and reduced velocity (G) compared to EV controls; see also Supplemental Video 3. Data in (B-E and G) are represented as mean±SD of n>49 cells from 3 independent experiments; *p<0.05 and ***p<0.001 using Kruskal-Wallis test with Dunn's multiple comparisons test (B-E) or one-way ANOVA with Tukey's multiple comparisons test (G); and data in (F) are represented as mean±SEM of n=60 cells from 3 independent experiments, ***p<0.001 using two-way ANOVA with Sidak's multiple comparisons test.

FIG. 7. OBSCN restoration suppresses breast cancer metastasis in vivo. (A) NSG female mice were injected with MDA-MB-231 EV, sgAS2, or sgAS3 expressing cells into the 4′ mammary gland, monitored over time for primary tumor growth, and euthanized at endpoint (i.e., primary tumor reached ˜1 cm³ in volume) for distant organ collection and evaluation; schematic was created with BioRender.com. (B-D) Quantification of hLINE levels by qPCR in the lungs (B), axillary lymph nodes (C) and liver (D) demonstrated the presence of minimal (if any) micrometastases in animals injected with sgAS2 and sgAS3 expressing MDA-MB-231 cells compared to EV controls. Of note, one lymph node sample was removed from the EV group during statistical evaluation because it was identified as a statistical outlier using the ROUT method, since it contained visible macrometastases and thus had an exceptionally high amount of hLINE DNA (i.e., 1,810,160 pg DNA per mg of lymph node tissue) compared to the other lymph node samples. (E-F) Representative images of lung sections from mice injected with EV, sgAS2, and sgAS3 expressing MDA-MB-231 cells stained with H&E (E) and an anti-mitochondrial antibody specifically detecting human mitochondria (F). Insets include low magnification images (1×) of the entire section while high magnification images (10×) of the marked areas are shown. (G) Model depicting the regulatory role of OBSCN-AS1 on OBSCN transcriptional activation; schematic was created with BioRender.com. Data in (B-D) are represented as mean±SEM, with *p<0.05 and **p<0.01 using Kruskal-Wallis test with Dunn's multiple comparisons (B and D) or one-way ANOVA with Fisher's LSD multiple comparisons test (C).

FIG. 8. CRISPR-activation of the OBSCN promoter leads to moderate OBSCN upregulation in breast cancer cells. (A) Visual depiction of the genomic location of sgOBSCN1-4 targeting the OBSCN promoter (region shown chr1:228,207,893-228,208,300); sgOBSCN1-4 target sequences are shown as color-shaded areas with sgOBSCN1 in blue, sgOBSCN2 in red, sgOBSCN3 in green, and sgOBSCN4 in pink. (B-C) RT-qPCR analysis of MDA-MB-231 (B) and Hs578T (C) cells transduced with sgOBSCN1-4 lentiviruses showed moderate to no upregulation of OBSCN mRNA expression. (D-E) The modest or lack of upregulation at the mRNA level was mirrored at the protein level in both MDA-MB-231(D) and Hs578T (E) transduced cells as determined by immunoblotting assays followed by densitometric evaluation. (F-G) RT-qPCR analysis of MDA-MB-231 (F) and Hs578T (G) cells transduced with sgOBSCN1-4 lentiviruses showed no significant change in OBSCN-AS1 variant 1 and 2 transcript levels compared to EV control cells. Data are represented as mean±SD of n=3 independent experiments; *p<0.05 and NS=non-significant using one-way ANOVA with Dunnett's multiple comparisons test.

FIG. 9. OBSCN-AS1 promoter activation leads to robust expression of OBSCN-AS1/OBSCN with negligible off-target transcriptional changes. (A-C) Expression analysis of genes in close genomic proximity to the OBSCN-AS1/OBSCN gene pair. RT-qPCR analysis of GUK1 (A), IBA57 (B), and TRIM11 (C) in MDA-MB-231 EV, sgAS2, and sgAS3 transduced cells showed no significant change of their transcript levels. Data represented as mean±SD of n=3 independent experiments; NS=non-significant using one-way ANOVA with Dunnett's multiple comparisons test. (D) Volcano plot displaying the global transcriptional changes identified via RNAseq between MDA-MB-231 cells transduced with sgAS2 and sgAS3 or EV. Each data point in the scatter plot represents a gene, with the log 2(fold change) of each gene shown on the x-axis and the −log 10(p-value) on the y-axis, as determined using the Wald test. Genes with a p-value <0.01 and a log 2 fold change >1 are indicated by red dots, representing up-regulated genes, while genes with a p-value <0.01 and a log 2 fold change <−1 are indicated by green dots, representing down-regulated genes. A total of 7 genes were found to be differentially expressed with OBSCN-AS1 and OBSCN displaying the highest significance and increased expression, while the remaining 5 exhibiting low significance and/or minimal fold change.

FIG. 10. OBSCN-AS1/OBSCN upregulation in Hs578T cells reduces cell migration but does not alter cell proliferation. (A) Hs578T cells transduced with sgAS2 show significantly reduced wound closure compared to EV control cells 9 h post-wound in wound healing assays. (B) Transwell migration of sgAS2 expressing Hs578T cells is markedly decreased compared to EV control cells. (C-H) Single cell migration analysis demonstrated that Hs578T cells expressing sgAS2 exhibit significantly reduced percent cell entry (C), increased cell entry time (D), unaltered longitudinal area (E), and decreased velocity (F), speed (G), and persistence (H) compared to EV control cells. See also supplemental video 2. (I) Cell proliferation of Hs578T cells expressing EV, sgAS2 or sgAS3 was unaffected, as determined by the number of living cells during a 96 h period. Data in (A-C) are represented as mean±SD of n=3 independent experiments, **p<0.01 and ***p<0.001 using two-tailed t test; data in (D-H) are represented as mean±SD of n 90 cells (D, and F-H) or n=52 cells (E) from 3 independent experiments; NS=non-significant, **p<0.01, and ***p<0.001 using two-tailed t test (D-E) or Mann-Whitney test (F-H); and data in (I) are represented as mean±SD of n=3 independent experiments; NS=non-significant using two-way ANOVA with Sidak's multiple comparisons test.

FIG. 11. Evaluation of primary tumors following injection of control and OBSCN-AS1/OBSCN expressing MDA-MB-231 cells. (A) Tumor volume measurements over time following implantation of MDA-MB-231 cells transduced with EV, sgAS2, or sgAS3 into the 4^{th} mammary gland of NSG female mice. Animals were euthanized at endpoint defined as the timepoint at which primary tumors reached ˜1 cm³ in volume; EV: ˜7 weeks, sgAS2: ˜4 weeks, and sgAS3: ˜6 weeks. Data are represented as mean±SEM of n=5 mice per group. (B) Representative images of primary tumors during dissection; tumors generated from EV control MDA-MB-231 cells often invaded into the peritoneum (yellow arrowhead points to a region where the tumor has spread into the surrounding tissue), while tumors generated from sgAS2 and sgAS3 MDA-MB-231 cells appear well encapsulated. (C) Representative image of macrometastases observed in the axillary lymph node of one of the EV control animals. (D) Quantification of the weight of primary tumors harvested post-euthanasia indicated no statistical difference between EV control and sgAS2 or sgAS3 groups; data are represented as mean±SEM of n=5 mice per group, NS=non-significant using Kruskal-Wallis test with Dunn's multiple comparisons. (E-F) Representative images (4× magnification) of lung sections from mice injected with EV, sgAS2, and sgAS3 expressing MDA-MB-231 cells stained with H&E (E) and anti-mitochondria antibody specifically detecting human mitochondria (F).

FIG. 12. Higher OBSCN levels correlate with increased breast cancer patient responsiveness to anthracyclines. Breast cancer patients' response to anthracyclines chemotherapy was assessed using the ROC plot analysis tool (rocplot.org), which indicated that OBSCN is a positive predictive biomarker with AUC=0.697 (p-value=7.2e-06).

FIG. 13. Representative original immunoblots, with red rectangles marking the depicted areas.

### DETAILED DESCRIPTION OF THE INVENTION

Mounting evidence has implicated the giant, cytoskeletal protein obscurin (720-870 kDa), encoded by the OBSCN gene, in the predisposition and development of breast cancer. Accordingly, prior work has shown that the sole loss of OBSCN from normal breast epithelial cells increases survival and chemoresistance, induces cytoskeletal alterations, enhances their migratory and invasive potentials, and promotes metastasis in the presence of oncogenic KRAS. Consistent with these observations, analysis of Kaplan-Meier Plotter data sets reveals that low OBSCN levels correlate with significantly reduced overall and relapse-free survival in breast cancer patients. Despite the compelling evidence implicating OBSCN loss in breast tumorigenesis and progression, its regulation has remained elusive, limiting any efforts to restore its expression, a major challenge given its molecular complexity and gigantic size (˜170 kb).

Herein, it is shown that OBSCN-AS1, a novel nuclear long-noncoding RNA (lncRNA) gene originating from the minus-strand of OBSCN, and OBSCN display positively correlated expression and are downregulated in breast cancer biopsies. OBSCN-AS1 regulates OBSCN expression through chromatin remodeling involving H3-lysine-4-trimethylation enrichment, associated with an open chromatin conformation, and RNA polymerase-II recruitment. CRISPR-activation of OBSCN-AS1 in triple negative breast cancer cells effectively and specifically restores OBSCN expression, and markedly suppresses cell migration, invasion, and dissemination from three-dimensional spheroids in vitro and metastasis in vivo. Collectively, these results reveal the previously unknown regulation of OBSCN by an antisense lncRNA and the metastasis suppressor function of the OBSCN-AS1/OBSCN gene pair, which may be of use as prognostic biomarkers and/or therapeutic targets for metastatic breast cancer.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

The term “sequence” relates to a nucleotide sequence of any length, which can be DNA or RNA; can be linear, circular or branched and can be either single-stranded or double stranded; and also can include an amino acid sequence of any length.

The term “identity” relates to an exact nucleotide-to-nucleotide or amino acid-to-amino acid correspondence of two polynucleotides or polypeptide sequences, respectively. Two or more sequences (polynucleotide or amino acid) can be compared by determining their percent identity. Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Sequence similarity” between polynucleotides can be determined by hybridization of polynucleotides under conditions that allow formation of stable duplexes between homologous regions, followed by digestion with single-stranded-specific nuclease(s), and size determination of the digested fragments.

The term “treating” or “treatment”, as used herein, means reversing, alleviating, inhibiting the progress of, or preventing the disorder or condition to which such term applies, or one or more symptoms of such disorder or condition.

A “therapeutically effective amount” or “effective amount” refers to a minimal amount of therapeutic agent which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder.

“Agent” or “therapeutic agent” refers to a chemical compound, small molecule, or other composition, such as a sgRNA, polypeptide such as CAS9 or variants thereof, antibody, protease inhibitor, hormone, chemokine or cytokine, capable of inducing a desired therapeutic or prophylactic effect when properly administered to a subject. For example, therapeutic agents for breast cancer include agents that prevent or inhibit development or metastasis of breast cancer, either acting alone, or in combination with other agents.

The terms “subject” and “patient” are used interchangeably herein, and refer to an animal such as a mammal. In general, the terms refer to a human. The terms also includes domestic animals bred for food, sport, or as pets, including horses, cows, sheep, poultry, fish, pigs, cats, dogs, and zoo animals, goats, apes (e.g. gorilla or chimpanzee), and rodents such as rats and mice. Typical subjects include persons susceptible to, suffering from or that have suffered from cancer.

In one embodiment, the invention provides a method for increasing OBSCN expression in a cell, comprising providing to the cell one or more agents that increases levels of OBSCN-AS1 lncRNA or a variant thereof in the cell.

In another embodiment, the invention provides a method of treating cancer in a subject, comprising administering to the subject an effective amount of one or more agents that increases levels of OBSCN-AS1 lncRNA or a variant thereof in cancer cells of the subject. In some embodiments, the treatment increases OBSCN expression and reduces cancer cell migration and/or metastasis.

Obscurins comprise a family of giant, multidomain, cytoskeletal proteins originally identified in striated muscles where they play key roles in their structural organization and contractile activity (Kontrogianni-Konstantopoulos et al., Journal of Muscle Research and Cell Motility 2005; 26: 419-426; Kontrogianni-Konstantopoulos et al., Physiol Rev 2009; 89: 1217-1267; Perry et al., IUBMB life 2013; 65: 479-486). (29, 31, 34). The human OBSCN gene spans 150 kb on chromosome 1q42 and undergoes extensive splicing to give rise to at least 4 isoforms (Fukuzawa et al., Journal of Muscle Research and Cell Motility 2005; 26: 427-434; Russell et al., Gene 2002; 282: 237-246) (19, 38). The prototypical form of obscurin, obscurin A, is about 720 kDa and contains multiple signaling and adhesion domains arranged in tandem (Kontrogianni-Konstantopoulos et al., Physiol Rev 2009; 89: 1217-1267). The NH₂-terminus of the molecule contains repetitive immunoglobulin (Ig) and fibronectin-III (Fn-III) domains, while the COOH-terminus includes several signaling domains, including an IQ motif, a src homology 3 (SH3) domain, a Rho-guanine nucleotide exchange factor (Rho-GEF), and a pleckstrin homology (PH) domain, interspersed by non-modular sequences. In addition to obscurin A, the OBSCN gene gives rise to another large isoform, obscurin B or giant (g) MLCK, which has a molecular mass of about 870 kDa (Fukuzawa et al., Journal of Muscle Research and Cell Motility 2005; 26: 427-434; Russell et al., Gene 2002; 282: 237-246). Obscurin B contains two serine/threonine kinase domains, which replace the non-modular COOH-terminus of obscurin A (Hu et al., FASEB J 2013; 27: 2001-2012). The two serine/threonine kinases may also be expressed independently as smaller isoforms, containing one (about 55 kDa) or both (about 145 kDa) kinase domains (Borisov et al., Journal of Cellular Biochemistry 2008; 103: 1621-1635). Obscurins are abundantly expressed in normal breast epithelial cells, where they localize at cell-cell junctions, the nucleus, and in cytoplasmic puncta coinciding with the Golgi membrane, but their expression is markedly diminished in breast cancer cells (Perry et al., FASEB J 2012; 26: 2764-2775).

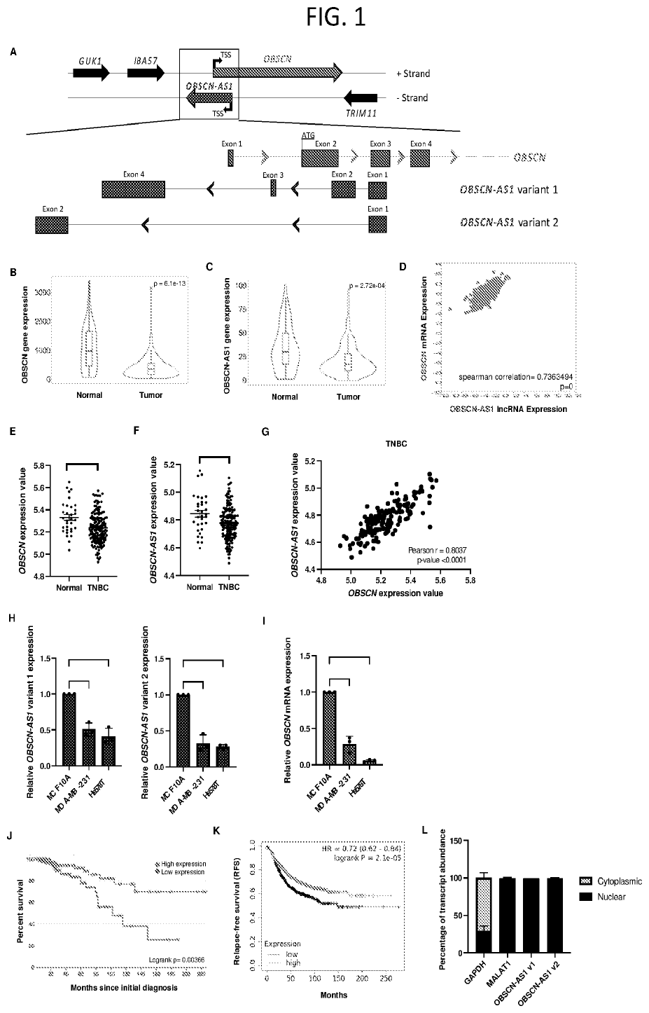

OBSCN-Antisense RNA 1 (OBSCN-AS1) is a lncRNA gene located in human chromosome 1q42.13 that originates from the minus strand of the protein-coding OBSCN gene (Guardia et al., Biochim Biophys Acta Rev Cancer 1876, (2021) 188567). Two splice variants of OBSCN-AS1 have been described with variant-1 (2884 nts; NCBI Reference Sequence: NR_073154.1; SEQ ID NO:72) consisting of 4 exons and variant-2 (981 nts; NR_073155.1; SEQ ID NO:73) containing 2 exons (FIG. 1A).

The type of cell for increasing expression of OBSCN is not limiting, and can include any type of cell where OBSCN is normally or not normally expressed. The cells can include cells in vivo, live isolated cells, for example, cultured cells, primary cells, or cells from an established cell line. In some embodiments, the cell is a cancerous cell, or a cell suspected of being or at risk of being cancerous. The type of cancer cell is not limiting. Cancer cells are often in the form of a tumor, but such cells may exist alone within an animal, may be a non-tumorigenic cancer cell, such as a leukemia cell, and also include ex vivo cells isolated from a subject or cells from cancer cell lines.

In some embodiments, the cell is a breast cancer cell. In some embodiments, the cell is a HER2-positive cancer cell. In some embodiments, the cell is a HER2 over-expressing or HER2 high-expressing cancer cell. In some embodiments, the cell is a HER2 low-expressing cancer cell. In some embodiments, the cell is a Her2-negative tumor or cancer cell. In some embodiments, the cancer cell is a triple-negative breast cancer cell (TNBC).

The cancer to be treated is not limiting. In some embodiments, the cancer is breast cancer. In some embodiments, the cancer is triple-negative breast cancer.

As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The amount of increase in expression of OBSCN that can be achieved by the methods herein is not limiting. In some embodiments, expression is increased by about 25%, about 50%, about 75%, about 2-fold, about 3-fold, about 4-fold, about 5-fold, about 10-fold, about 15-fold, about 20-fold, about 30-fold, about 40-fold, or more, in the cells.

The amount of increase in the level of OBSCN-AS1 lncRNA is not necessarily limiting, provided it is sufficient to increase the expression level of OBSCN in a cell. In some embodiments, the OBSCN-AS1 lncRNA is increased by about 2-fold, about 3-fold, about 4-fold, about 5-fold, about 10-fold, about 15-fold, about 20-fold, about 30-fold, about 40-fold, about 50-fold, about 75-fold, about 100-fold or more, in the cells.

In some embodiments, the OBSCN-AS1 lncRNA is selected from OBSCN-AS1 lncRNA variant 1, OBSCN-AS1 lncRNA variant 2 and a combination thereof.

In some embodiments, the one or more agents comprises a nucleic acid encoding OBSCN-AS1 lncRNA or a variant thereof. The nucleic acid to be delivered to the cell or subject can comprise DNA or RNA. In some embodiments, the OBSCN-AS1 lncRNA is encoded by SEQ ID NO:72, SEQ ID NO:73, or both. Variants include nucleic acids that are at least 60%, 65%, 70%, 75%, 80%, 85%, 90%, 91%, 92%, 93%, 94%, 95%, 96%, 97%, 98%, 99% identical to SEQ ID NO:72 or SEQ ID NO:73. The term “identity” relates to an exact nucleotide-to-nucleotide or amino acid-to-amino acid correspondence of two polynucleotides or polypeptide sequences, respectively. Variants also encompass fragments of OBSCN-AS1 lncRNA, including fragments that are not 100% identical across SEQ ID NO:72 or SEQ ID NO:73. In some embodiments, fragments of SEQ ID NO:72 are at least 1500, 1600, 1700, 1800, 1900, 2000, 2100, 2200, 2300, or 2400 nucleotides in length, and at least 60%, 65%, 70%, 75%, 80%, 85%, 90%, 91%, 92%, 93%, 94%, 95%, 96%, 97%, 98%, 99% identical to SEQ ID NO:72 over that same span of sequence. In some embodiments, fragments of SEQ ID NO:73 are at least 500, 600, 700, 800, 900, 950, 960, or 970 nucleotides in length, and at least 60%, 65%, 70%, 75%, 80%, 85%, 90%, 91%, 92%, 93%, 94%, 95%, 96%, 97%, 98%, 99% identical to SEQ ID NO:73 over that same span of sequence.

In some embodiments, endogenous expression of OBSCN-AS1 lncRNA is increased by the one or more agents. In some embodiments, the one or more agents binds to a promoter region of OBSCN-AS1 and increases expression of OBSCN-AS1 lncRNA in the cell. In some embodiments, the one or more agents that is administered comprises a CRISPR/Cas system comprising i) a nucleic acid encoding a sgRNA comprising a targeting domain which is complementary with a target sequence of the OBSCN-AS1 gene and ii) a nucleic acid encoding a Cas9 polypeptide or a variant thereof.

A “target sequence” is a nucleic acid sequence that defines a general region of a nucleic acid to which a binding molecule may bind, provided sufficient conditions for binding exist. Herein, the target domain is a sgRNA sequence, and the target sequence corresponds to the sequence on the OBSCN-AS1 gene to which the target domain of the sgRNA binds.

The Cas9 polypeptide or variant thereof is not limiting provided it increases expression of OBSCN-AS1. In some embodiments, the Cas9 polypeptide is a variant that is nuclease deficient (dCas9). In some embodiments, the Cas9 polypeptide variant is fused to one or more polypeptide sequences capable of activating transcription and/or modifying histones. In some embodiments, the one or more polypeptide sequences comprises an amino acid sequence from VP64, VP192, CBP, p300 or a combination thereof. In some embodiments, a CRISPR/dCas9 Synergistic Activation Mediator (SAM) lentiviral system can be used to activate expression of OBSCN-AS1 lncRNA (Konermann et al., Nature 517, (2015), 583-588; Joung et al., Nat Protoc 12, (2017), 828-863), which is incorporated by reference in its entirety.

In some embodiments, the dCas9 has an amino acid sequence of SEQ ID NO:74.

In some embodiments, the dCas9 is fused to an amino acid sequence of VP64. In some embodiments, the VP64 amino acid sequence comprises SEQ ID NO:75.

In some embodiments, the invention provides a nucleic acid encoding a sgRNA that is compatible for use with a Cas9 or variant molecule, wherein the sgRNA comprises a targeting domain which is complementary with a target sequence of OBSCN-AS1, preferably a sequence in or nearby a promoter.

In some embodiments, the CRISPR/Cas system is provided to the cell by one or more vectors. In some embodiments, the CRISPR/Cas system is provided to the cell by a virus. In some embodiments, the virus is an adeno-associated virus (AAV), a lentivirus, a retrovirus or a combination thereof. In some embodiments, the vector is a lentiviral vector.

In some embodiments, the CRISPR/Cas system comprises a first vector encoding i) a nuclease deficient Cas9 fused to one or more polypeptide sequences capable of activating transcription and/or modifying histones and ii) the sgRNA. In some embodiments, the sgRNA comprises two MS2 loops. In some embodiments, the sequence of the MS2 loops is encoded by SEQ ID NO:76.

In some embodiments, the CRISPR/Cas system comprises a second vector, wherein the second vector encodes MS2 coat protein fused to p65 and HSF-1 activation domains.

In some embodiments, the first vector backbone (to be used to insert the specific targeting domain sequence) is commercially available as LentiSAMv2 (Addgene #75112). This vector backbone is available in addgene and was generated and described in the following publication: Genome-scale CRISPR-Cas9 knockout and transcriptional activation screening. Joung et al., Nat Protoc. 2017 April; 12(4):828-863. doi: 10.1038/nprot.2017.016. Epub 2017 Mar. 23. 10.1038/nprot.2017.016 PubMed 28333914. This vector contains the dCas9-VP64 fusion, MS2 loops at tetraloop and stemloop 2, and contains the BsmBI enzyme sites for insertion of desired sgRNA spacer sequence. All the sequences of the components of the vectors can be found in addgene (https://www.addgene.org/75112/sequences/). The vector has a sequence of SEQ ID NO:77.

A sgRNA molecule, as that term is used herein, refers to a nucleic acid that promotes the specific targeting or homing of a sgRNA molecule/Cas9 molecule (or variant such as a nuclease deficient Cas9) complex to a target nucleic acid. As set forth herein, the target nucleic acid is a OBSCN-AS1 gene. The sgRNA molecule/Cas9 (or variant) molecule complex effects expression of OBSCN-AS1 lncRNA, thereby promoting expression of OBSCN in the cells or subject.

The sgRNA molecule can be unimolecular (having a single RNA molecule), sometimes referred to herein as “chimeric” sgRNAs, or modular (comprising more than one, and typically two, separate RNA molecules). In one embodiment, the sgRNA molecule can be used with a Cas9 protein or variant from Staphylococcus aureus.

The sgRNA comprises a targeting domain (which is complementary to the target nucleic acid) and other sequences that are necessary to bind Cas9. The targeting domain comprises a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the sgRNA molecule will comprise the base thymine (T). While not wishing to be bound by theory, it is believed that the complementarity of the targeting domain with the target sequence contributes to specificity of the interaction of the sgRNA molecule/Cas9 (or variant) molecule complex with a target nucleic acid. It is understood that in a targeting domain and target sequence pair, the uracil bases in the targeting domain will pair with the adenine bases in the target sequence. In an embodiment, the target domain itself comprises, in the 5′ to 3′ direction, an optional secondary domain, and a core domain. In an embodiment, the core domain is fully complementary with the target sequence. In an embodiment, the targeting domain is 5 to 50, 10 to 40, e.g., 10 to 30, e.g., 15 to 30, e.g., 15 to 25 nucleotides in length. In an embodiment, the targeting domain is 15, 16, 17, 18, 19, 20, 21, 22, 23, 24 or 25 nucleotides in length. The strand of the target nucleic acid with which the targeting domain is complementary is referred to herein as the complementary strand. Some or all of the nucleotides of the domain can have a modification, e.g., a modification described herein. Guidance on the selection of targeting domains can be found, e.g., in Fu et al., Nat Biotechnol 2014 (doi: 10.1038/nbt.2808) and Sternberg S H et al., Nature 2014 (doi: 10.1038/nature13011).

In some embodiments, the sgRNA comprises a targeting domain which is complementary with a target sequence which comprises any one or a combination of SEQ ID NO:13; SEQ ID NO:16; SEQ ID NO:19; and SEQ ID NO:22.

In some embodiments, the targeting domain is 10 to 100, 10 to 90, 10 to 80, 10 to 70, 10 to 60, 10 to 50, 10 to 40, 10 to 30, 10 to 20 or 10 to 15 nucleotides in length. In other embodiments, the targeting domain is 20 to 100, 20 to 90, 20 to 80, 20 to 70, 20 to 60, 20 to 50, 20 to 40, 20 to 30, or 20 to 25 nucleotides in length. In some embodiments, the targeting domain is 16, 17, 18, 19, 20, 21, 22, 23, 24 or 25 nucleotides in length.

In some embodiments, the targeting domain has full complementarity with the target sequence. In some embodiments, the targeting domain has or includes 1, 2, 3, 4, 5, 6, 7 or 8 nucleotides that are not complementary with the corresponding nucleotide of the targeting domain.

In some embodiments, the target domain includes 1, 2, 3, 4 or 5 nucleotides that are complementary with the corresponding nucleotide of the targeting domain within 5 nucleotides of its 5′ end. In an embodiment, the target domain includes 1, 2, 3, 4 or 5 nucleotides that are complementary with the corresponding nucleotide of the targeting domain within 5 nucleotides of its 3′ end.

In some embodiments, the target domain includes 1, 2, 3, or 4 nucleotides that are not complementary with the corresponding nucleotide of the targeting domain within 5 nucleotides of its 5′ end. In some embodiments, the target domain includes 1, 2, 3, or 4 nucleotides that are not complementary with the corresponding nucleotide of the targeting domain within 5 nucleotides of its 3′ end.

In some embodiments, the degree of complementarity, together with other properties of the sgRNA, is sufficient to allow targeting of a Cas9 molecule to the targeted gene.

In some embodiments, the targeting domain comprises two consecutive nucleotides that are not complementary to the target domain (“non-complementary nucleotides”), e.g., two consecutive noncomplementary nucleotides that are within 5 nucleotides of the 5′ end of the targeting domain, within 5 nucleotides of the 3′ end of the targeting domain, or more than 5 nucleotides away from one or both ends of the targeting domain.

In some embodiments, no two consecutive nucleotides within 5 nucleotides of the 5′ end of the targeting domain, within 5 nucleotides of the 3′ end of the targeting domain, or within a region that is more than 5 nucleotides away from one or both ends of the targeting domain, are not complementary to the targeting domain.

In some embodiments, there are no noncomplementary nucleotides within 5 nucleotides of the 5′ end of the targeting domain, within 5 nucleotides of the 3′ end of the targeting domain, or within a region that is more than 5′ nucleotides away from one or both ends of the targeting domain.

In some embodiments, the targeting domain comprises one or more modifications, e.g., modifications that render it less susceptible to degradation or more bio-compatible, e.g., less immunogenic. By way of example, the backbone of the targeting domain can be modified with a phosphorothioate. In one embodiment, a nucleotide of the targeting domain can comprise a 2′ modification (e.g., a modification at the 2′ position on ribose), e.g., a 2′ acetylation, e.g., a 2′ methylation, or other modification.

Methods for designing sgRNAs are described herein, including methods for selecting, designing and validating target domains. Targeting domains discussed herein can be incorporated into the sgRNAs described herein. Methods for selection and validation of target sequences as well as off-target analyses are described, e.g., Mali et al., 2013 Science 339(6121): 823-826; Hsu et al., 2013 Nat Biotechnol, 31(9): 827-32; Fu et al., 2014 Nat Biotechnol, doi: 10.1038/nbt.2808. PubMed PMID: 24463574; Heigwer et al., 2014 Nat Methods 11(2):122-3. doi: 10.1038/nmeth.2812. PubMed PMID: 24481216; Bae et al., 2014 Bioinformatics PubMed PMID: 24463181; Xiao A et al., 2014 Bioinformatics PubMed PMID: 24389662.

For example, a software tool can be used to optimize the choice of sgRNA within a user's target sequence, e.g., to minimize total off-target activity across the genome. Off target activity may be other than cleavage. For each possible sgRNA choice, e.g., using S. pyogenes Cas9, the tool can identify all off-target sequences (e.g., preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate sgRNA molecules can be evaluated by art-known methods.

Cas molecules and variants, particularly nuclease deficient variants of a variety of species can be used in the methods and compositions described herein. In some embodiments, the Cas9 or variant is from Staphylococcus aureus. In some embodiments, the Cas9 or variant is from S. pyogenes, S. thermophiles, or Neisseria meningitides. Additional Cas9 species include: Acidovorax avenae, Actinobacillus pleuropneumoniae. Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis. Streptococcus sp., Subdoligranulum sp., Tistrella mobilis, Treponema sp., or Verminephmrobacter eiseniae.

A Cas9 or variant molecule, as that term is used herein, refers to a molecule that can interact with a sgRNA molecule and, in concert with the sgRNA molecule, localize (e.g., target or home) to a site which comprises a target domain.

Exemplary naturally occurring Cas9 molecules are described in Chylinski et al., RNA Biology 2013; 10:5, 727-737, which is incorporated herein by reference. These molecules can be modified to create nuclease deficient variants. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g.; strain F0211), S. agalactiae (e.g., strain NEM316, A909), Listeria monocytogenes (e.g., strain F6854), Listeria innocua (L. innocua, e.g., strain Clip11262) Enterococcus italicus (e.g., strain DSM 15952), or Enterococcus faecium (e.g., strain 1,231,408). Additional exemplary Cas9 molecules are a Cas9 molecule of Neisseria meningitidis (Hou et al. PNAS Early Edition 2013, 1-6) and a S. aureus Cas9 molecule.

Cas9 molecules with desired properties can be made in a number of ways, e.g., by alteration of a parental, naturally occurring Cas9 molecule to provide an altered Cas9 molecule having a desired property. One or more mutations or differences relative to a parental Cas9 molecule can be introduced. Such mutations and differences can comprise: substitutions (e.g., conservative substitutions or substitutions of non-essential amino acids); insertions; or deletions. In some embodiments, a Cas9 molecule can comprises one or more mutations or differences, e.g., at least 1, 2, 3, 4, 5, 10, 15, 20, 30, 40 or 50 mutations but less than 200, 100, or 80 mutations relative to a reference Cas9 molecule.

Candidate Cas9 molecules, candidate sgRNA molecules, candidate Cas9 molecule/sgRNA molecule complexes, can be evaluated by art-known methods or as described herein. For example, exemplary methods for evaluating the endonuclease activity of Cas9 molecule are described, e.g., in Jinek et al., Science 2012; 337(6096):816-821.

### Combination Therapy

In some embodiments, the subject is administered one or more additional therapeutic agents or treatments. The additional therapeutic agent or treatment is not limiting. In some embodiments, the one or more additional therapeutic agents or treatments are those commonly used to treat cancer. In some embodiments, the subject is administered one or more additional anti-cancer agents, surgery and/or radiotherapy in combination with the one or more agents that increase the levels of OBSCN AS1 lncRNA herein.

In some embodiments, the additional therapeutic agent comprises a chemotherapeutic agent. In some embodiments, the therapeutic agent is an anthracycline chemotherapeutic agent.

In some embodiments, the additional therapeutic agent is an anti-cancer agent selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and I 131 Iodine Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine) Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine¹³¹ Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nivolumab, Nolvadex (Tamoxifen Citrate), Nplate (Romiplostim), Obinutuzumab, OEPA, Ofatumumab, OFF, Olaparib, Omacetaxine Mepesuccinate, Oncaspar (Pegaspargase), Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and I 131 Iodine Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

In some embodiments, the anti-cancer agent is an immunotherapeutic agent. The cancer immunotherapy is not limiting and can include one or more immunotherapies. There are several different approaches to immunotherapy. For example, immunotherapies can include monoclonal antibodies, checkpoint inhibitors/immune modulators, therapeutic cancer vaccines, oncolytic viruses, adoptive T cell transfer, cytokines, and adjuvant immunotherapy.

In certain embodiments, the combination of therapeutic agents discussed herein may be administered concurrently as a single composition in a pharmaceutically acceptable carrier, or concurrently as separate compositions with each agent in a pharmaceutically acceptable carrier. In another embodiment, the combination of therapeutic agents can be administered sequentially. The duration of time separating administrations in sequential administrations is not necessarily limiting.

In some embodiments, the additional therapeutic agent comprises purified tumor antigens (including recombinant proteins, peptides, and carbohydrate molecules), cells, and cells transfected with genes encoding immune stimulating cytokines (He et al. (2004) J. Immunol. 173:4919-28).

The additional therapeutic agent can include a cancer vaccine. Many experimental strategies for vaccination against tumors have been devised (see Rosenberg, S. (2000) Development of Cancer Vaccines, ASCO Educational Book Spring: 60-62; Logothetis, C., 2000, ASCO Educational Book Spring: 300-302; Khayat, D. (2000) ASCO Educational Book Spring: 414-428; Foon, K. (2000) ASCO Educational Book Spring: 730-738; see also Restifo and Sznol, Cancer Vaccines, Ch. 61, pp. 3023-3043 in DeVita et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The study of gene expression and large scale gene expression patterns in various tumors has led to the definition of so called tumor specific antigens (Rosenberg (1999) Immunity 10:281-7). In many cases, these tumor specific antigens are differentiation antigens expressed in the tumors and in the cell from which the tumor arose. More importantly, many of these antigens can be shown to be the targets of tumor specific T cells found in the host. In certain embodiments, the subject is administered one or more recombinant proteins and/or peptides expressed in a tumor in order to generate an immune response to these proteins. These proteins are normally viewed by the immune system as self-antigens and are, therefore, tolerant to them. The tumor antigen may also include the protein telomerase, which is required for the synthesis of telomeres of chromosomes and which is expressed in more than 85% of human cancers and in only a limited number of somatic tissues (Kim et al. (1994) Science 266: 2011-2013). These somatic tissues may be protected from immune attack by various means. Tumor antigen may also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al. (1998) Nature Medicine 4: 328-332). DCs may also be transduced by genetic means to express these tumor antigens as well. DCs have also been fused directly to tumor cells for the purposes of immunization (Kugler et al. (2000) Nature Medicine 6:332-336). As a method of vaccination, DC immunization may be effectively further combined with the one or more therapeutic agents described herein.

Non-limiting examples of tumor vaccines that can also be used include peptides possible head and neck cancer antigens, such as p53, melanoma-associated antigens (MAGEs) such as MAGE-3, NY-ESO-1, cyclin B1, caspase-8, SART-1, carcino-embryonal antigen, and extracellular matrix metalloproteinase inducer (EMMPRIN) (CD147). The peptides can be coupled with antigen presenting cells, such as dendritic cells in some embodiments.

The one or more therapeutic agents herein can also be used in combination with bispecific antibodies that target Fcα or Fcγ receptor-expressing effector cells to tumor cells (see, e.g., U.S. Pat. Nos. 5,922,845 and 5,837,243). Bispecific antibodies can be used to target two separate antigens. For example anti-Fc receptor/anti-tumor antigen (e.g., Her-2/neu) bispecific antibodies have been used to target macrophages to sites of tumor. This targeting may more effectively activate tumor specific responses. The T cell arm of these responses could be augmented by therapeutic agents described herein. In some embodiments, antigen may be delivered directly to DCs by the use of bispecific antibodies which bind to tumor antigen and a dendritic cell specific cell surface marker.

In another embodiment, the additional therapeutic agent can comprise anti-neoplastic antibodies, such as Rituxan® (rituximab), Herceptin® (trastuzumab), Bexxar® (tositumomab), Zevalin® (ibritumomab), Campath® (alemtuzumab), Lymphocide® (eprtuzumab), Avastin® (bevacizumab), and Tarceva® (erlotinib), Kadcyla® (ado-trastuzumab emtansine), Perjeta® (pertuzumab), Adcetris® (brentuximab vedotin), Erbitux® (cetuximab), Vectibix® (panitumumab), Gazyva® (obinutuzumab), Arzerra® (ofatumumab), Cyramza® (ramucirumab), Blincyto® (blinatumomab) nimotuzumab, panitumumab, zalutumumab, cetuximab, matuzumab, figitumumab, bavituximab, Ch14.18, and rilotumumab. In some embodiments, the antibody can be bound to a toxin. By way of example and not wishing to be bound by theory, treatment with an anti-cancer antibody or an anti-cancer antibody conjugated to a toxin can lead to cancer cell death (e.g., tumor cells).

Tumors evade host immune surveillance by a large variety of mechanisms. Many of these mechanisms may be overcome by the inactivation of proteins, which are expressed by the tumors and which are immunosuppressive. These include, among others, TGF-β (Kehrl, J. et al. (1986) J Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). In another example, antibodies to one or more of these entities can be administered in combination with the therapy herein.

In another embodiment, the therapeutic agents can be used in combination with one or more checkpoint inhibitors or immune modulators. Checkpoint inhibitors/immune modulators can make cancer cells more susceptible to attack by the immune system. Checkpoint inhibitors and immune modulators include CTLA-4 inhibitors such as Yervoy® and tremelimumab, PD-1/PD-L1 inhibitors such as Keytruda®, Opdivo®, MPDL3280A and MEDI4736, LAG-3 inhibitors and KIR inhibitors. In some embodiments, the immune modulator is selected from CD27 inhibitors and GITR inhibitors.

Other antibodies that may be used to activate host immune responsiveness can be further used in combination with the therapies herein. These include molecules on the surface of dendritic cells that activate DC function and antigen presentation. Anti-CD40 antibodies are able to substitute effectively for T cell helper activity (Ridge, J. et al. (1998) Nature 393: 474-478). Activating antibodies to T cell costimulatory molecules, such as OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) may also provide for increased levels of T cell activation.

In some embodiments, the subject is administered T cells. There are also several treatment protocols that involve ex vivo activation and expansion of antigen specific T cells and adoptive transfer of these cells into recipients in order to generate antigen-specific T cells against tumor. Adoptive T cell transfer is an anti-cancer approach that enhances the natural cancer-fighting ability of the body's T cells by removing immune system cells, growing and/or making changes to them outside of the body, and then re-infusing them back into the patient. In some embodiments, T cells can be collected from a sample of a patient's tumor and multiplied in a laboratory. In some embodiments, T cells can be taken out of the body and genetically modified to attack antigens on cancer cells. In some embodiments, T cells can be taken out of the body and equipped with special receptors called chimeric antigen receptors (CARs); when given back to the patient, these “CAR T cells” recognize and attack cancer cells.

In some embodiments, the additional therapeutic agent comprises an oncolytic virus. An oncolytic virus is virus that can activate a greater immune response.

In some embodiments, the additional therapeutic agent comprises one or more cytokines. In some embodiments, the cytokine is selected from IL-2 and IFN-alpha.

In some embodiments, the additional therapy comprises a standard cancer treatment, such as chemotherapeutic regimes. In some embodiments, it may be possible to reduce the dose of the chemotherapeutic reagent administered (Mokyr et al. (1998) Cancer Research 58: 5301-5304). In some embodiments, chemotherapeutic compounds should result in increased levels of tumor antigen in the antigen presentation pathway as a result of increased cell death. Other combination therapies that can be employed include radiation, surgery, or hormone deprivation.

### Vectors, Delivery Compositions and Host Cells

The mode of delivering the one or more agents useful in the methods herein is not limiting. In some embodiments, the nucleic acids can be administered to the subject either as naked nucleic acid, e.g., in conjunction with a delivery reagent such as a lipid nanoparticle, or as a recombinant plasmid or viral vector that expresses the nucleic acids. Delivery of nucleic acids or vectors to an individual may occur by any suitable means, e.g., using a cyclodextrin delivery system; ionizable lipids; DPC conjugates; GalNAc-conjugates; or polymeric nanoparticles made of low-molecular-weight polyamines and lipids (see Kanasty et al. Nature Materials 12, 967-977 (2013) for general review of same).

In some embodiments, the invention provides vectors that comprise nucleic acids that are useful in carrying out the methods herein, including OBSCN-AS1 and/or the CRISPR/Cas9 system of the present invention, and host cells which are genetically engineered with vectors of the invention and the production of polypeptides and nucleic acids of the invention by recombinant techniques. Cell-free translation systems can also be employed to produce such proteins using RNAs derived from the constructs of the invention.

Representative examples of appropriate hosts include bacterial cells, such as streptococci, staphylococci, E. coli, Streptonmyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, and 293 cells; and plant cells. A great variety of expression systems can be used, including DNA or RNA vectors.

The components for genetically modifying the cell can be delivered, formulated, or administered in a variety of forms. When a component is delivered encoded in nucleic acid the nucleic acid will typically include a control region, e.g., comprising a promoter, to effect expression. In some embodiments, useful promoters for Cas9 or variant molecule sequences include CMV, EF-1a, MSCV, PGK, CAG control promoters. In some embodiments, useful promoters for sgRNAs include H1, EF-1a and U6 promoters. Promoters with similar or dissimilar strengths can be selected to tune the expression of components. Sequences encoding a Cas9 or variant molecule can comprise a nuclear localization signal (NLS), e.g., an SV40 NLS. In some embodiments, a promoter for a Cas9 or variant molecule or a gRNA molecule can be, independently, inducible, tissue specific, or cell specific.

Nucleic acid encoding Cas9 (or variants) and/or sgRNA molecules can be administered to subjects or delivered into cells by art-known methods or as described herein. For example, Cas9 or variant encoding and/or sgRNA-encoding DNA can be delivered by vectors (e.g., viral or non-viral vectors), non-vector based methods (e.g., using naked DNA or DNA complexes), or a combination thereof.

In some embodiments, the Cas9 or variant and one or more sgRNAs are located on a single nucleic acid molecule. In some embodiments, the Cas9 or variant and one or more sgRNAs are located on separate nucleic acid molecules. In some embodiments, wherein multiple sgRNAs are utilized, the Cas9 or variant and one or more sgRNAs are located on a single nucleic acid molecule and one or more additional sgRNAs are located a different nucleic acid molecule.

In some embodiments, the Cas9 or variant and/or sgRNA-encoding nucleic acid is delivered by a vector such as a viral vector/virus or plasmid. In some embodiments, a vector can comprise a sequence that encodes a Cas9 or variant molecule and/or a sgRNA molecule. In some embodiments, a vector can also comprise a sequence encoding a signal peptide (e.g., for nuclear localization, nucleolar localization, mitochondrial localization), fused, e.g., to a Cas9 or variant molecule sequence. For example, a vector can comprise a nuclear localization sequence (e.g., from SV40) fused to the sequence encoding the Cas9 or variant molecule.

In some embodiments, one or more regulatory/control elements, e.g., a promoter, an enhancer, an intron, a polyadenylation signal, a Kozak consensus sequence, internal ribosome entry sites (IRES), a 2A sequence, and a splice acceptor or donor can be included in the vectors. In some embodiments, the promoter is recognized by RNA polymerase II (e.g., a CMV promoter). In other embodiments, the promoter is recognized by RNA polymerase III (e.g., a U6 promoter). In some embodiments, the promoter is a regulated promoter (e.g., inducible promoter). In other embodiments, the promoter is a constitutive promoter. In some embodiments, the promoter is a tissue specific promoter. In some embodiments, the promoter is a viral promoter. In other embodiments, the promoter is a non-viral promoter.

The particular viral vector that can be used is not particularly limiting. In some embodiments, the viral vector will typically comprise a highly attenuated, non-replicative virus. Viral vectors include, but are not limited to, DNA viral vectors such as those based on adenoviruses, herpes simplex virus, avian viruses, such as Newcastle disease virus, poxviruses such as vaccinia virus, and parvoviruses, including adeno-associated virus; and RNA viral vectors, including, but not limited to, the retroviral vectors. Vaccinia vectors and methods useful in immunization protocols are described in U.S. Pat. No. 4,722,848. Retroviral vectors include murine leukemia virus, and lentiviruses such as human immunodeficiency virus. Naldini et al. (1996) Science 272:263-267. Replication-defective retroviral vectors harboring a nucleotide sequence of interest as part of the retroviral genome can be used. Such vectors have been described in detail. (Miller et al. (1990) Mol. Cell. Biol. 10:4239; Kolberg, R. (1992) J. NIH Res. 4:43; Cornetta et al. (1991) Hum. Gene Therapy 2:215).

Adenovirus and adeno-associated virus vectors useful in the invention may be produced according to methods already taught in the art. (See, e.g., Karlsson et al. (1986) EMBO 5:2377; Carter (1992) Current Opinion in Biotechnology 3:533-539; Muzcyzka (1992) Current Top. Microbiol. Immunol. 158:97-129; Gene Targeting: A Practical Approach (1992) ed. A. L. Joyner, Oxford University Press, NY). Several different approaches are feasible.

Alpha virus vectors, such as Venezuelan Equine Encephalitis (VEE) virus, Semliki Forest virus (SFV) and Sindbis virus vectors, can be used for efficient gene delivery. Replication-deficient vectors are available. Such vectors can be administered through any of a variety of means known in the art, such as, for example, intranasally or intratumorally. See Lundstrom, Curr. Gene Ther. 2001 1:19-29.

Additional literature describing viral vectors which could be used in the methods of the present invention include the following: Horwitz, M. S., Adenoviridae and Their Replication, in Fields, B., et al. (eds.) Virology, Vol. 2, Raven Press New York, pp. 1679-1721, 1990); Graham, F. et al., pp. 109-128 in Methods in Molecular Biology, Vol. 7: Gene Transfer and Expression Protocols, Murray, E. (ed.), Humana Press, Clifton, N.J. (1991); Miller, et al. (1995) FASEB Journal 9:190-199, Schreier (1994) Pharmaceutica Acta Helvetiae 68:145-159; Schneider and French (1993) Circulation 88:1937-1942; Curiel, et al. (1992) Human Gene Therapy 3:147-154; WO 95/00655; WO 95/16772; WO 95/23867; WO 94/26914; WO 95/02697 (Jan. 26, 1995); and WO 95/25071.

In some embodiments, the viral vector is a retrovirus/lentivirus, adenovirus, adeno-associated virus, alpha virus, vaccinia virus or a herpes simplex virus. In some embodiments, the viral vector is a lentiviral vector. One or more viral vectors can be used to deliver the one or more therapeutic agents, e.g., the CRISPR/Cas9 system herein.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in humans. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the Cas9 or variant molecule and/or the sgRNA molecule. In other embodiments, the virus causes long-lasting, e.g., at least 1 week, 2 weeks, 1 month, 2 months, 3 months, 6 months, 9 months, 1 year, 2 years, or permanent expression, of the Cas9 or variant molecule and/or the sgRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the Cas9 or variant and/or sgRNA-encoding nucleic is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the Cas9 or variant and/or sgRNA-encoding nucleic acid is delivered by a recombinant lentivirus. In some embodiments, the lentivirus is replication-defective and does not comprise one or more genes required for viral replication.

In some embodiments, the Cas9 or variant and/or sgRNA-encoding nucleic acid is delivered by a recombinant adenovirus. In some embodiments, the adenovirus is engineered to have reduced immunity in human.

In some embodiments, the Cas9 or variant and/or sgRNA-encoding nucleic acid is delivered by a recombinant AAV. In some embodiments, the AAV can incorporate its genome into that of a host cell, e.g., a vascular smooth muscle cell. In some embodiments, the AAV is a self-complementary adeno-associated virus (scAAV), e.g., a scAAV that packages both strands which anneal together to form double stranded DNA. AAV serotypes that can be used in the methods of the invention include, e.g., AAV1, AAV2, modified AAV2 (e.g., modifications at Y444F, Y500F, Y730F and/or S662V), AAV3, modified AAV3 (e.g., modifications at Y705F, Y731 F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods.

In some embodiments, the Cas9 or variant and/or sgRNA-encoding nucleic acid is delivered by a hybrid virus, e.g., a hybrid of one or more of the viruses described herein.

In some embodiments, a packaging cell can be used to form a virus particle that is capable of infecting a host or target cell. Such a cell can include a 293 cell, which can package adenovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed. For example, an AAV vector used in gene therapy typically only possesses inverted terminal repeat (ITR) sequences from the AAV genome which are required for packaging and gene expression in the host or target cell. The missing viral functions can be supplied in trans by the packaging cell line. The viral nucleic acid can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the Cas9 or variant and/or sgRNA-encoding nucleic is delivered by a non-vector based method (e.g., using naked DNA or DNA complexes). For example, the nucleic acid can be delivered by organically modified silica or silicate (Ormosil), electroporation, gene gun, sonoporation, magnetofection, lipid-mediated transfection, dendrimers, inorganic nanoparticles, calcium phosphates, or a combination thereof.

In some embodiments, the Cas9 or variant and/or sgRNA-encoding nucleic acid is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, the Cas9 or variant molecule and the sgRNA molecule are delivered by different modes, or as sometimes referred to herein as differential mode. Different or differential modes, as used herein, refer modes of delivery that confer different pharmacodynamic or pharmacokinetic properties on the subject component molecule, e.g., a Cas9 or variant molecule or sgRNA molecule. For example, the modes of delivery can result in different tissue distribution, different half-life, or different temporal distribution, e.g., in a selected compartment, tissue, or organ.

In some embodiments, suitable delivery reagents for administration in conjunction with the present nucleic acids or vectors include the Mirus Transit TKO lipophilic reagent; lipofectin; lipofectamine; cellfectin; or polycations (e.g., polylysine), or liposomes. In some embodiments, a particular delivery reagent comprises a liposome.

Liposomes can aid in the delivery of the present nucleic acids or vectors to a particular tissue, and can also increase the blood half-life of the nucleic acids. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

In some embodiments, liposomes or nanoparticles encapsulating the present nucleic acids comprise a ligand molecule that can target the liposomes or nanoparticles to a particular cell or tissue at or near the site of interest. Ligands that bind to receptors prevalent in the tissues to be targeted, such as monoclonal antibodies that bind to surface antigens, are contemplated. In particular cases, the liposomes or nanoparticles are modified so as to avoid clearance by the mononuclear macrophage and reticuloendothelial systems, for example by having opsonization-inhibition moieties bound to the surface of the structure. In one embodiment, a liposome or nanoparticle of the invention can comprise both opsonization-inhibition moieties and a ligand. Opsonization-inhibiting moieties for use in preparing the liposomes or nanoparticles of the disclosure are typically large hydrophilic polymers that are bound to the liposome membrane. As used herein, an opsonization inhibiting moiety is “bound” to a liposome or nanoparticle when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes or nanoparticles by the macrophage-monocyte system (“MMS”) and reticuloendothelial system (“RES”); e.g., as described in U.S. Pat. No. 4,920,016, the entire disclosure of which is herein incorporated by reference. Liposomes or nanoparticles modified with opsonization-inhibition moieties thus remain in the circulation much longer than unmodified liposomes.

Stealth liposomes or nanoparticles are known to accumulate in tissues fed by porous or “leaky” microvasculature. Thus, target tissue characterized by such microvasculature defects, for example solid tumors, will efficiently accumulate these liposomes; see Gabizon, et al. (1988), P.N.A.S., USA, 18: 6949-53. In addition, the reduced uptake by the RES lowers the toxicity of stealth liposomes by preventing significant accumulation in the liver and spleen. Thus, liposomes or nanoparticles of the invention that are modified with opsonization-inhibition moieties can deliver the present nucleic acids to tumor cells.

Opsonization inhibiting moieties suitable for modifying liposomes or nanoparticles are preferably water-soluble polymers with a number-average molecular weight from about 500 to about 40,000 Daltons, and more preferably from about 2,000 to about 20,000 Daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments, the opsonization-inhibiting moiety is a PEG, PPG, or derivatives thereof. Liposomes modified with PEG or PEG-derivatives are sometimes called “PEGylated liposomes.” The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH 3 and a solvent mixture such as tetrahydrofuran and water in a 30:12 ratio at 60 degrees Celcius.

The nucleic acids can be administered using recombinant plasmids. Such recombinant plasmids can also be administered directly or in conjunction with a suitable delivery reagent, including the Mirus Transit LT 1 lipophilic reagent; lipofectin; lipofectamine; cellfectin; polycations (e.g., polylysine) or liposomes.

The one or more therapeutic agents for increasing levels of OBSCN-AS1 lncRNA can be administered to the subject by any suitable means. For example, the agents can be administered by gene gun, electroporation, or by other suitable parenteral or enteral administration routes, or by injection, for example, by intramuscular or intravenous injection. Suitable parenteral administration routes include intravascular administration (e.g. intravenous bolus injection, intravenous infusion, intra-arterial bolus injection, intra-arterial infusion and catheter instillation into the vasculature); peri- and intra-tissue administration (e.g., peri-tumoral and intra-tumoral injection, intra-retinal injection or subretinal injection); subcutaneous injection or deposition including subcutaneous infusion (such as by osmotic pumps); direct (e.g., topical) application to the area at or near the site of interest, for example by a catheter or other placement device (e.g., a corneal pellet or a suppository, eye-dropper, or an implant comprising a porous, non-porous, or gelatinous material); and inhalation. In a particular embodiment, injections or infusions of the composition(s) are given at or near the site of disease.

The one or more agents for increasing levels of OBSCN-AS1 lncRNA can be administered in a single dose or in multiple doses. Where the administration of a composition is by infusion, the infusion can be a single sustained dose or can be delivered by multiple infusions. Injection of the agent directly into the tissue is at or near the site of need. Multiple injections of the agent into the tissue at or near the site of interest are encompassed within this disclosure.

One skilled in the art can also readily determine an appropriate dosage regimen for administering the one or more agents for increasing levels of OBSCN-AS1 lncRNA of the invention to a given subject. For example, the composition(s) can be administered to the subject once, such as by a single injection or deposition at or near the site of interest. In some embodiments, the composition(s) can be administered to a subject once or twice daily to a subject once weekly for a period of from about three to about twenty-eight days, in some embodiments, from about seven to about ten weeks. In some dosage regimens, the composition(s) is injected at or near the site of interest once a day for seven days. Where a dosage regimen comprises multiple administrations, it is understood that the effective amount of composition(s) administered to the subject can comprise the total amount of composition(s) administered over the entire dosage regimen.

In some embodiments, the nucleic acids, e.g., the CRISPR/Cas system is provided to the cell by one or more vectors. In some embodiments, the cell is provided a first vector encoding i) a nuclease deficient Cas9 fused to one or more polypeptide sequences capable of activating transcription and/or modifying histones and ii) the sgRNA.

In some embodiments, the sgRNA comprises two MS2 loops. In some embodiments, the cell is provided a second vector, wherein the second vector encodes MS2 coat protein fused to p65 and HSF-1 activation domains.

### Compositions

In another embodiment, the invention provides pharmaceutical compositions capable of increasing levels of OBSCN AS1 lncRNA or variants thereof in cells.

The pharmaceutical compositions can be formulated according to known methods for preparing pharmaceutically acceptable useful compositions, and may include a pharmaceutically acceptable carrier. The carrier may be liquid, solid, or semi-solid for example. Formulations are described in a number of sources which are well known to those of skill in the art. The physical and/or chemical characteristics of compositions of the inventions may be modified or optimized according to skill in the art, depending on the mode of administration. The compositions may be in any suitable form, depending on the desired method of administration.

The pharmaceutical composition may be adapted for administration by any appropriate route, for example by the oral, rectal, nasal, topical, vaginal or parenteral routes.

Pharmaceutical compositions adapted for parenteral administration include aqueous and non-aqueous sterile injection solutions which may contain anti-oxidants, buffers, bacteriostats, and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in freeze-dried conditions requiring only the addition of a sterile liquid immediately prior to use. Extemporaneous injection solutions and suspensions may be prepared from sterile powders, granules, and tablets. The pharmaceutical compositions may contain preserving agents, solubilizing agents, stabilizing agents, wetting agents, emulsifiers, salts, buffers, antioxidants, etc.

In some embodiments, the pharmaceutical composition comprises one or more components of a CRISPR/Cas9 system as described herein. In some embodiments, the composition comprises a nucleic acid encoding a sgRNA comprising a targeting domain which is complementary with a target sequence of the OBSCN-AS1 gene and a Cas9 polypeptide or a variant thereof. In some embodiments, the Cas9 polypeptide variant is nuclease deficient (dCas9) and is fused to one or more polypeptide sequences capable of activating transcription and/or modifying histones. In some embodiments, the one or more polypeptide sequences comprises an amino acid sequence from VP64, VP192, CBP, p300 or a combination thereof. In some embodiments, the target sequence of the OBSCN-AS1 gene is selected from the group consisting of SEQ ID NO:13; SEQ ID NO:16; SEQ ID NO:19; and SEQ ID NO:22.

In some embodiments, the composition can comprise one or more viral vectors. In some embodiments, the viral vector is an adeno-associated virus (AAV), a lentivirus, a retrovirus or a combination thereof.

In some embodiments, the composition comprises a first vector encoding i) a nuclease deficient Cas9 fused to one or more polypeptide sequences capable of activating transcription and/or modifying histones and ii) the sgRNA. In some embodiments, the sgRNA comprises two MS2 loops. In some embodiments, the composition further comprises a second vector, wherein the second vector encodes MS2 coat protein fused to p65 and HSF-1 activation domains. In some embodiments, a first composition comprises the first vector, and a second composition comprises the second vector. The compositions can be administered concurrently or sequentially.

### Cancer Prognosis

In another embodiment, the invention provides a method of prognosing cancer in a subject, comprising
i) providing cancer cells or tissue from the subject;
ii) assaying the cells or tissue for expression of OBSCN and comparing OBSCN expression level to a control; and
iii) assaying the cells or tissue for expression of OBSCN-AS1 and comparing OBSCN-AS1 expression level to a control;
wherein reduced expression level of OBSCN and/or OBSCN-AS1 relative to the control indicate an increased probability for metastasis, wherein normal or increased expression level of OBSCN and/or OBSCN-AS1 relative to the control indicate an increased sensitivity to an anthracycline chemotherapeutic agent.

“Prognosis” refers to a prediction of the course of a disease, such as breast cancer. The prediction can include, e.g., determining the likelihood of a subject to develop metastatic disease, to survive a particular amount of time (e.g. determine the likelihood that a subject will survive 1, 2, 3, 4, or 5 years), to respond to a particular therapy (e.g., chemotherapy), or combinations thereof.

Detection or measurement of expression levels is performed as compared to controls, which may include, but are not limited to, a comparison with data from normal subjects and/or comparable normal tissue (in the same or different subjects) absent the disease or disorder present in the subject (or the specific tissue of the subject tested). In some embodiments, the comparison may be between levels detected at a variety of time intervals (and/or locations) in a patient. In some embodiments, the detection needs to be statistically significant as compared to background or control levels; the ability to assess significance is well-known in the art.

In some embodiments, the methods of prognosis further comprise administering an effective amount of a therapeutic agent to treat cancer. In some embodiments, the subject is administered an effective amount of an anthracycline chemotherapeutic agent. In some embodiments, the subject is administered an effective amount of the CRISPR/Cas system to increase expression of OBSCN and/or OBSCN-AS1.

Any cells or tissue suspected of containing or lacking the cancer markers described herein may be tested according to methods of embodiments of the present invention. By way of non-limiting examples, the sample may be tissue (e.g., breast tissue obtained by biopsy).

In some embodiments, the cells or tissue are from a tumor sample. The term “tumor sample” means any tissue tumor sample derived from the patient. The tissue sample is obtained for the purpose of the in vitro evaluation. The sample can be fresh, frozen, fixed (e.g., formalin fixed), or embedded (e.g., paraffin embedded). In a particular embodiment the tumor sample may result from the tumor resected from the patient. In another embodiment, the tumor sample may result from a biopsy performed in the primary tumor of the patient or performed in a metastatic sample distant from the primary tumor of the patient. For example an endoscopical biopsy performed in the bowel of the patient affected by a colorectal cancer.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. OBSCN Restoration Via OBSCN-AS1 Long-Noncoding RNA CRISPR-Targeting Suppresses Metastasis in Triple Negative Breast Cancer

While advances have been made in the detection and treatment of primary breast tumors, metastasis and recurrence have remained major challenges. Mounting evidence implicates OBSCN in breast tumorigenesis. Accordingly, low(er) OBSCN levels correlate with significantly reduced overall and relapse-free survival in breast cancer patients, while high(er) OBSCN levels correlate with increased patient-responsiveness to anthracycline-chemotherapy. Restoration of OBSCN expression could therefore be of high pathophysiological relevance. This example unravels mechanistic information involving the direct regulation of OBSCN via OBSCN-AS1, a nuclear long-noncoding RNA gene. Remarkably, as described herein, OBSCN restoration via OBSCN-AS1 targeting drastically suppresses cell migration and metastasis. Collectively, our study pinpoints the metastasis suppressor function of the OBSCN-AS1/OBSCN pair that may serve as prognostic biomarker and/or therapeutic target for metastatic breast cancer.

### Results

### Expression Profile of OBSCN-AS1 and OBSCN in Breast Cancer

OBSCN-Antisense RNA 1 (OBSCN-AS1) is a novel gene encoding an antisense lncRNA transcribed from the complementary strand of the OBSCN gene, encoding the giant cytoskeletal proteins obscurins, located in human chromosome 1q42.13 (FIG. 1A). OBSCN-AS1 gives rise to two lncRNA transcript variants that share partial complementarity with the OBSCN protein-coding transcripts (FIG. 1A). To evaluate the noncoding nature of OBSCN-AS1, we used the Coding Potential Calculator 2 bioinformatics tool (http://cpc2.gao-lab.org) and found that OBSCN-AS1 variant 1 and variant 2 were classified as noncoding sequences with coding probabilities of 0.164208 and 0.180045, respectively. Conversely, the OBSCN mRNA sequence was classified as a coding sequence with a coding probability of 1. While previous experimental and bioinformatics studies have implicated OBSCN loss in breast tumorigenesis and metastasis, the role of OBSCN-AS1 has been elusive (Guardia et al., Biochim Biophys Acta Rev Cancer 1876, (2021) 188567; Rajendran et al., Oncotarget 8, (2017), 102263-102276). To gain insights on the potential involvement of OBSCN-AS1 in breast cancer, we analyzed RNA-seq data from breast invasive carcinoma patients available through the TNM plot database (TNMplot.com) (Bartha et al., Int J Mol Sci 22, (2021)). Our analysis revealed that similarly to OBSCN (FIG. 1B), OBSCN-AS1 expression is significantly reduced (FIG. 1C) in breast tumors compared to normal adjacent tissue, with the expression profile of the two genes exhibiting a positive correlation (FIG. 1D). Next, we analyzed the OBSCN and OBSCN-AS1 expression levels and correlation using transcriptome microarray data from 164 TNBC, a highly aggressive breast cancer subtype, and 32 normal breast tissue samples (Zhou et al., J Immunother Cancer 9, (2021)). In agreement with our subtype-independent findings, the expression of OBSCN and OBSCN-AS1 is significantly decreased in TNBC compared to normal breast tissue samples (FIG. 1E-F), and their expression is positively correlated (FIG. 1G).

To further investigate the expression profile of OBSCN-AS1 and OBSCN in breast cancer cell lines, we performed RT-qPCR using the non-tumorigenic breast epithelial cell line, MCF10A, and the TNBC cell lines, MDA-MB-231 and Hs578T, which are highly tumorigenic and metastatic in vivo (Yankaskas et al., Nat Biomed Eng 3, (2019), 452-465). Consistent with the RNA-seq analysis of breast cancer tumor biopsies, our findings indicated that both MDA-MB-231 and Hs578T cell lines contained significantly reduced levels of OBSCN-AS1 lncRNA variant 1 and 2 transcripts (FIG. 1H) and OBSCN mRNA (FIG. 1I) compared to MCF10A cells. Considering that decreased levels of OBSCN have been associated with drastically reduced overall (FIG. 1J; Betastasis.com) and relapse-free [FIG. 1K; kmplot.com; (Gyorffy et al., Comput Struct Biotechnol J 19, (2021), 4101-4109)] survival, we postulated that the concomitant loss of OBSCN and OBSCN-AS1 may be of pathophysiological relevance.

Consistent with this notion, lncRNAs have been shown to have important transcriptional, post-transcriptional and translational roles, and their cellular localization is a strong indicator of their mechanism of action (Fernandes et al., Noncoding RNA, (2019), 5). We therefore proceeded to assess the cellular localization of OBSCN-AS1 in non-tumorigenic breast epithelial MCF10A cells via cellular fractionation followed by RT-qPCR. Our studies indicated that both OBSCN-AS1 lncRNA variants 1 and 2 exhibit a nearly exclusive nuclear distribution (FIG. 1L); the validity of this finding was corroborated by concomitant evaluation of the distribution of well-known marker genes with MALAT1, a lncRNA coding gene, showing predominant accumulation in the nucleus, as previously reported, and GAPDH, a protein-coding gene, preferentially found in the cytoplasm where it is translated into protein (Bernard et al., EMBO J 29, (2010), 3082-3093).

Taken together, these findings indicate that the expression profiles of OBSCN and OBSCN-AS1 are interrelated in breast cancer epithelial cells and suggest that the nuclear-enriched OBSCN-AS1 lncRNA may play a key role in the transcriptional regulation of OBSCN.

### Identification of the OBSCN and OBSCN-AS1 Promoter Regions in Breast Epithelial Cells

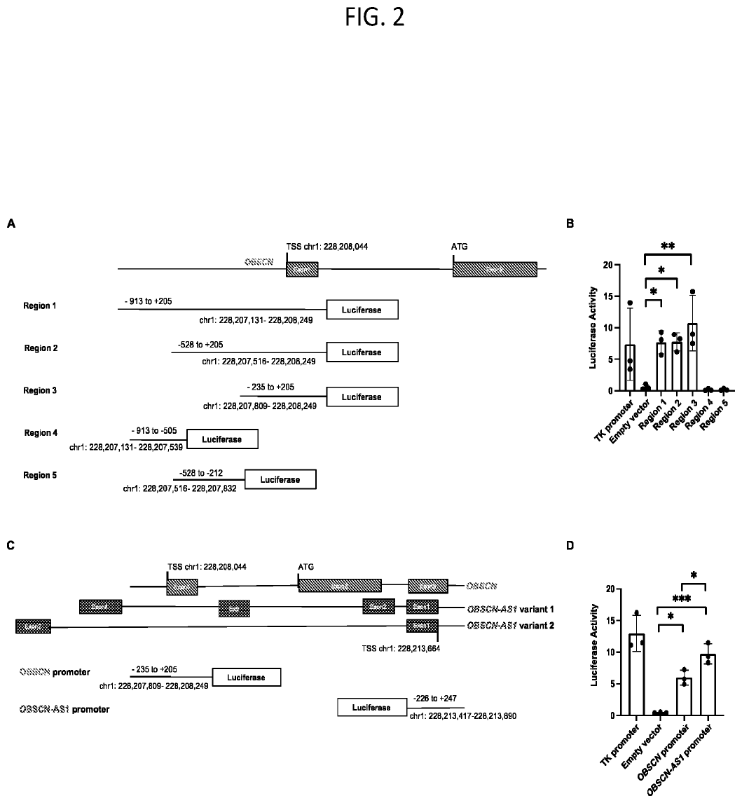

To gain insights into the transcriptional regulation of OBSCN, likely mediated by OBSCN-AS1, we first set out to experimentally identify the OBSCN and OBSCN-AS1 promoter regions, which have not been previously characterized. To do so, we generated a series of luciferase constructs containing overlapping or consecutive segments upstream of the OBSCN transcription start site (TSS), residing within exon 1, and a portion of the proximal 5′ untranslated region (FIG. 2A; regions 1-5 spanning −913 bp to +205 bp). These were used in dual luciferase reporter assays following transient transfection in MCF10A cells. Constructs including regions 1-3 induced a robust luciferase signal, with region 3 eliciting the highest response (FIG. 2B). Importantly, regions 4 and 5 failed to elicit any luciferase activity (FIG. 2B), indicating that the region included between −235 bp to +205 bp from the TSS contains key regulatory elements that modulate OBSCN activation. This finding is consistent with in silico analysis of the purported OBSCN promoter using the ENCODE Screen Registry V3, suggesting that the region encompassing −242 bp to +107 bp may contain “promoter-like” elements.

Next, we proceeded to identify the OBSCN-AS1 promoter region by generating a luciferase construct containing a region flanking the TSS present in exon 1 that encompasses −226 bp to +247 bp (FIG. 2C), which we again tested in a dual luciferase assay following transient transfection in MCF10A cells. Our results showed that the indicated construct exhibited robust luciferase activity (FIG. 2D), in agreement with bioinformatics analysis using ENCODE Screen Registry V3 that assigned a “promoter-like” signature to a region including −203 bp to +146 bp. Interestingly, parallel comparison of the OBSCN and OBSCN-AS1 promoter regions revealed that the latter displayed significantly higher luciferase activity (FIG. 2D), suggesting that the OBSCN-AS1 promoter may exhibit a stronger transcriptional signature.

Collectively, our findings experimentally define the proximal promoter regions of OBSCN (chr1:228,207,809-228,208,249) and OBSCN-AS1 (chr1:228,213,417-228,213,890) in breast epithelial cells.

### CRISPR-Activation of the OBSCN Promoter Leads to Moderate OBSCN Upregulation in Breast Cancer Cells

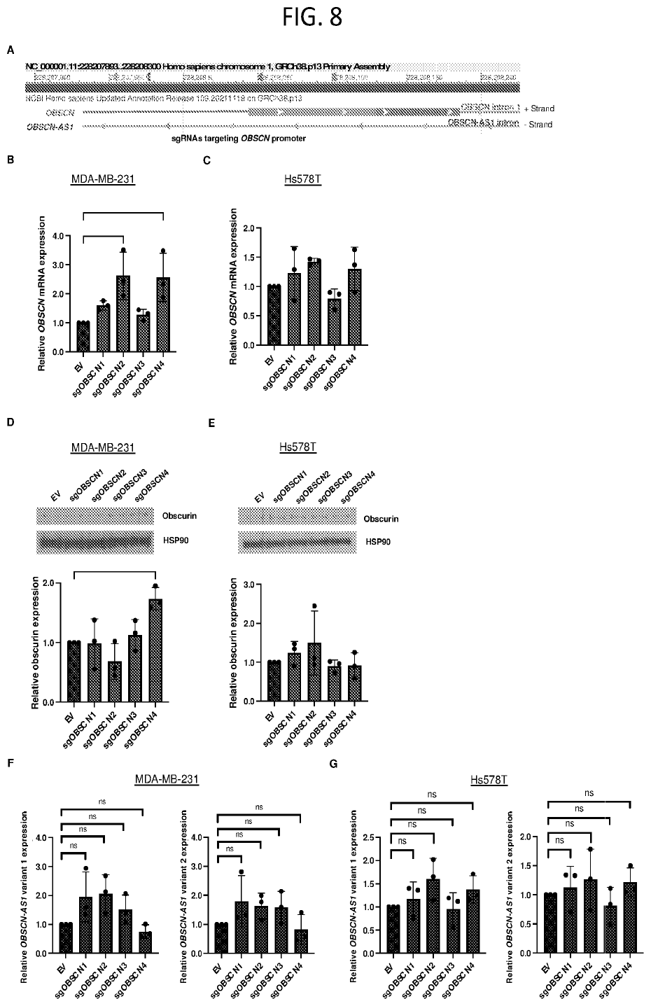

Our earlier work has implicated OBSCN loss in the potentiation of tumor growth and metastasis (Shriver et al., Oncogene 34, (2015), 4248-4259; Tuntithavornwat et al., Cancer Lett 526, (2022), 155-167). We therefore hypothesized that restoration of OBSCN expression and functionality in breast cancer cells may be beneficial. To test this hypothesis, we set forth to induce endogenous OBSCN expression in the TNBC MDA-MB-231 and Hs578T cell lines, which have reduced basal levels of obscurin transcripts (FIG. 1F), by targeting the OBSCN promoter via CRISPR-mediated activation. Specifically, we utilized the CRISPR/dCas9 Synergistic Activation Mediator (SAM) lentiviral system to examine the effectiveness of four single guide RNAs (sgOBSCN1-4) targeting distinct sequences within the OBSCN promoter (FIG. 8A) (Konermann et al., Nature 517, (2015), 583-588; Joung et al., Nat Protoc 12, (2017), 828-863). Interestingly, activation of the OBSCN promoter led to statistically significant, yet modest, upregulation of obscurin transcripts in MDA-MB-231 cells, ranging between 1.6-2.6 fold (FIG. 8B), and only an upward trend in Hs578T cells (FIG. 8C). These findings were further substantiated by immunoblotting analysis that revealed no significant upregulation in the expression of obscurin protein, except in MDA-MB-231 cells transduced with sgOBSCN4 that exhibited a ˜1.9-fold increase compared to control cells transduced with empty vector (EV) (FIG. 8D-E). Of note, we observed no significant changes in the transcript levels of OBSCN-AS1 variants 1 and 2 in sgOBSCN1-4 MDA-MB-231 and Hs578T transduced cells compared to EV control (FIG. 8F-G).

Together, these observations indicate that CRISPR-activation of the OBSCN promoter is not sufficient to induce robust transcription of OBSCN, at least under the current experimental conditions, underscoring the possible complexity of its regulation in breast cancer cells.

### CRISPR-Activation of the OBSCN-AS1 Promoter Leads to Robust Upregulation of OBSCN-AS1 and OBSCN in Breast Cancer Cells

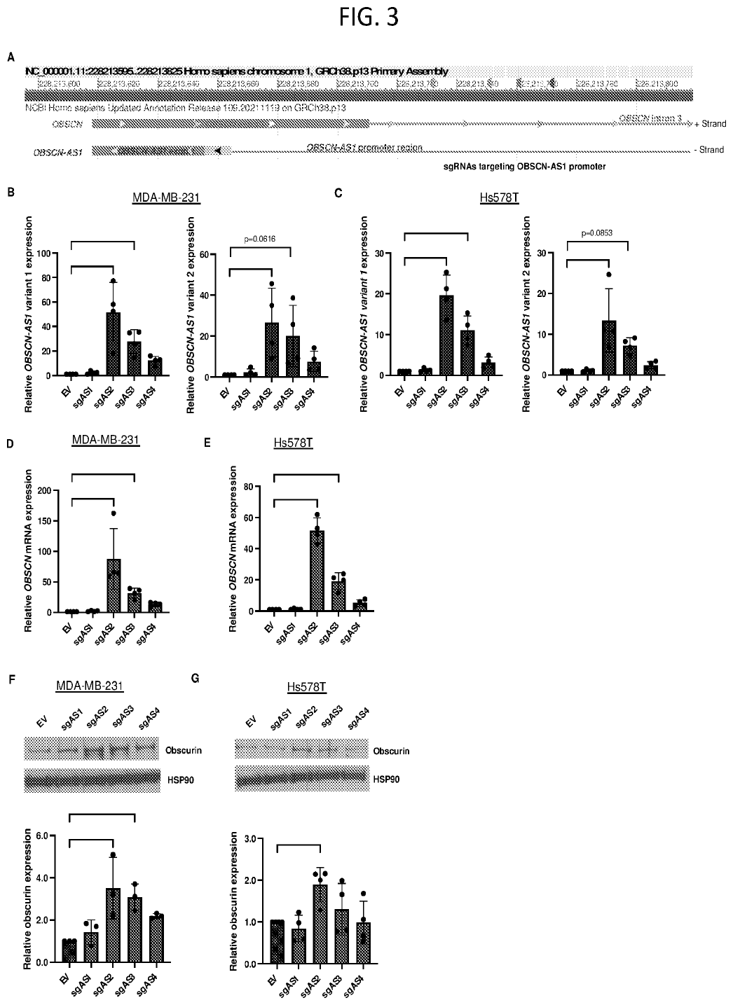

Given OBSCN-AS1's genomic location, nuclear localization, and correlated expression with OBSCN, we examined whether OBSCN-AS1 may regulate OBSCN transcriptional activation. To assess this possibility, we used the CRISPR/dCas9-SAM lentiviral system to test the effectiveness of four single guide RNAs (sgAS1-4) selectively targeting the promoter region of OBSCN-AS1 in MDA-MB-231 and Hs578T cells (FIG. 3A). CRISPR-activation of the OBSCN-AS1 promoter resulted in robust upregulation of OBSCN-AS1 lncRNA variant 1 and 2 transcripts ranging between 26.6-51.6 and 11-19.6 fold in MDA-MB-231 (FIG. 3B) and Hs578T (FIG. 3C) cells, respectively. Remarkably, CRISPR-targeting of the OBSCN-AS1 promoter also resulted in significant upregulation of the OBSCN mRNA in both TNBC cell lines tested, ranging between 31.8-87.8 fold in MDA-MB-231 (FIG. 3D) and 19.2-51.7 fold in Hs578T (FIG. 3E) cells. This substantial upregulation at the mRNA level was followed by a significant increase at the protein level, too, ranging between 3.1-3.5 and ˜1.9 fold in MDA-MB-231 and Hs578T cells, respectively (FIG. 3F-G), indicating that OBSCN-AS1 positively regulates OBSCN expression. Of note, a non-linear relationship between mRNA and protein expression levels has been postulated, and especially for giant proteins, like obscurin, with different factors influencing translational efficiency, including RNA secondary structure, ribosomal density and occupancy, codon bias etc (Maier et al., FEBS Lett 583, (2009), 3966-3973).

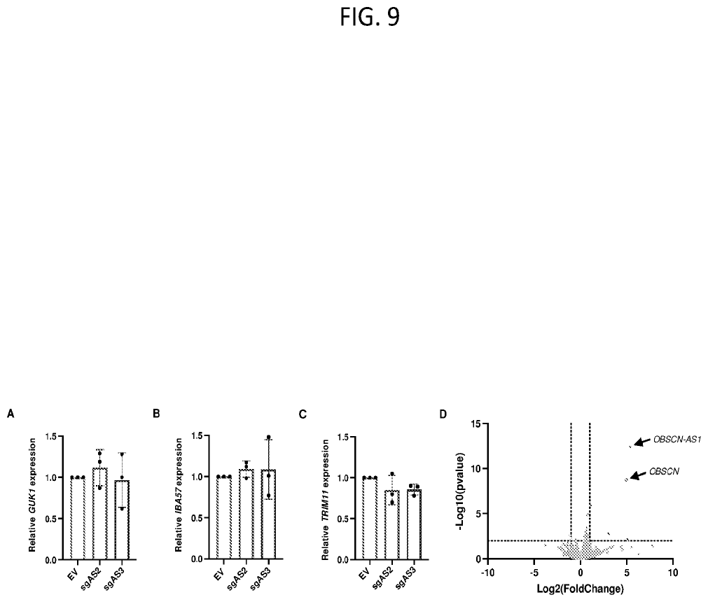

To determine the specificity of the CRISPR-mediated activation of the OBSCN-AS1 promoter, we quantified the expression of genes located proximally to OBSCN-AS1 and OBSCN (FIG. 1A) via RT-qPCR in MDA-MB-231 cells transduced with sgAS2 and sgAS3 that show the highest levels of OBSCN-AS1 and OBSCN upregulation. Our findings revealed no change in the transcript levels of GUK1, IBA57, and TRIM11 in sgAS2 or sgAS3 transduced MDA-MB-231 cells compared to EV controls (FIG. 9A-C). These findings were further corroborated by strand-specific RNA-seq experiments that examined genome-wide transcriptional changes between sgAS2 and sgAS3 activated and control MDA-MB-231 cells. A group of 7 genes were identified as differentially expressed (i.e., genes with an absolute log 2(fold change) >1 and p-value <0.01; FIG. 9D and Table 1), with the top 2 genes being OBSCN-AS1 and OBSCN, which exhibited the highest significance values and showed increased expression with a log 2(fold change) of 5.38 and 4.97, respectively (FIG. 9D and Table 1). The remaining 5 genes had low significance values and/or showed minimal fold changes (FIG. 9D and Table 1). Thus, the significant upregulation of OBSCN-AS1 and OBSCN expression and the minimal alterations in global gene transcription demonstrate the specificity of our CRISPR engineering approach and further indicate that OBSCN-AS1 plays a specific and direct role in OBSCN transcriptional regulation.

TABLE 1
Differentially expressed genes determined
by strand-specific RNA-seq.

| | | −Log10(p- | |
|---|---|---|---|
| Gene Name | Gene ID | value) | Log2(FoldChange) |
| OBSCN-AS1 | ENSG00000162913 | 12.42 | 5.38 |
| OBSCN | ENSG00000154358 | 8.74 | 4.97 |
| PSG11 | ENSG00000243130 | 5.96 | 1.10 |
| FRG2EP | ENSG00000282842 | 2.79 | 3.06 |
| AC134772.1 | ENSG00000244380 | 2.51 | −1.18 |
| ENO3 | ENSG00000108515 | 2.24 | −1.01 |
| CES1P2 | ENSG00000260765 | 2.11 | 5.04 |

### OBSCN-AS1 is a Positive Regulator of OBSCN Transcription Via Chromatin Remodeling

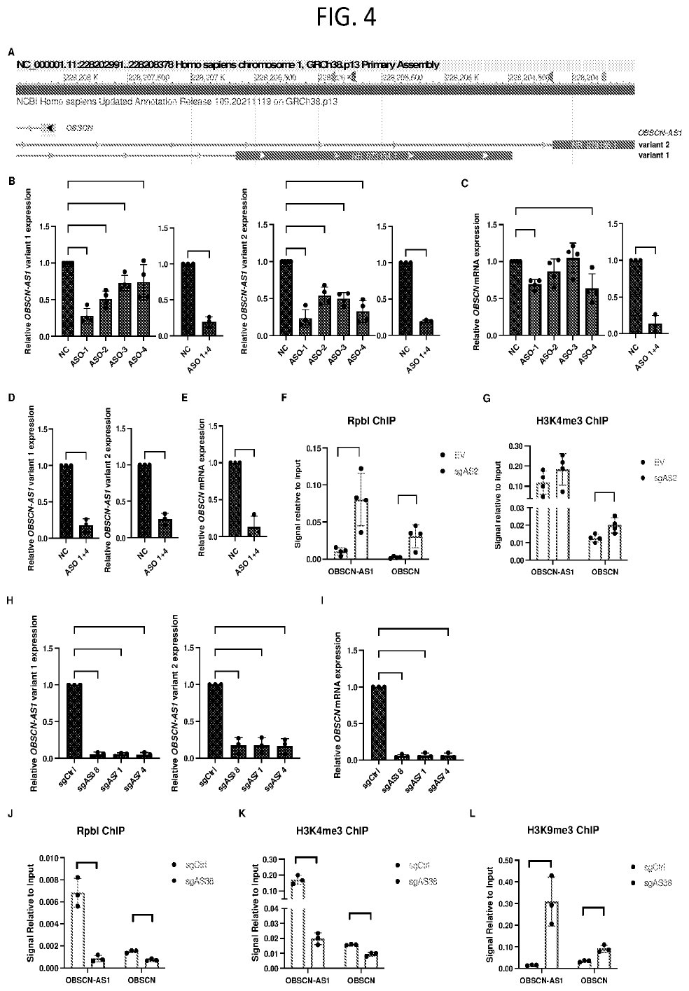

To assess whether OBSCN transcriptional activation via OBSCN-AS1 CRISPR-targeting is mediated by the OBSCN-AS1 lncRNA, we proceeded to knockdown OBSCN-AS1 in sgAS2 transduced MDA-MB-231 cells using antisense oligonucleotides (ASOs). ASOs were designed to target exon regions within OBSCN-AS1 variants 1 (i.e., ASO-1 and ASO-2) and 2 (i.e., ASO-3 and ASO-4), but not the OBSCN transcript (FIG. 4A). Of note, ASO-1 and ASO-2 localize within an intron region of variant 2 (FIG. 4A), and could therefore target variant 2, too, as ASOs may act on nascent (pre-spliced) in addition to mature (spliced) transcripts (Lai et al., Mol Cell 77, (2020), 1032-1043 e1034; Lee et al., Mol Cell 77, (2020), 1044-1054 e1043). ASO-treated cells exhibited statistically reduced transcript levels of variant 1 and/or 2, ranging between 27-77%, with enhanced knockdown, ˜81%, achieved in cells treated with a combination of the two most effective ASOs (i.e., ASO-1 and ASO-4; FIG. 4B). OBSCN mRNA expression was also statistically decreased (31-37%) in cells transfected with individual ASOs that induced the greatest knockdown of either both variants 1 and 2 (i.e., ASO-1) or variant 2 (ASO-4) (FIG. 4C). Notably, downregulation of OBSCN transcripts was markedly pronounced (˜87%) in cells transfected with both ASO-1 and ASO-4 (FIG. 4C). Consistent with our findings with sgAS2 transduced MDA-MB-231 cells, MCF10A cells treated with ASO-1 and ASO-4 exhibited significantly decreased levels of OBSCN-AS1 variant 1 (˜82%) and variant 2 (˜75%), and consequent downregulation of OBSCN mRNA (˜87%) (FIG. 4D-E), further substantiating that OBSCN-AS1 lncRNA plays an essential role in OBSCN transcriptional activation.

Nuclear lncRNAs have been shown to regulate gene expression through modulation of chromatin structure by RNA-mediated neutralization of positively charged histone tails (Dueva et al., Cell Chem Biol 26, (2019), 1436-1449 e1435). We therefore reasoned that nuclear OBSCN-AS1 lncRNA transcripts may regulate OBSCN expression through chromatin remodeling. To investigate this possibility, we measured the levels of H3 lysine 4 trimethylation (H3K4me3), a histone modification associated with active promoter conformation, and RNA polymerase II binding linked to enhanced transcription, by chromatin immunoprecipitation followed by qPCR (ChIP-qPCR). As expected, we found a marked increase of RpbI (the largest subunit of RNA polymerase II) occupancy at the OBSCN-AS1 promoter in sgAS2 transduced MDA-MB-231 cells, consistent with our CRISPR-activation approach (FIG. 4F). More importantly, we further observed a statistically significant enrichment of RpbI binding and H3K4me3 levels at the OBSCN promoter (FIG. 4F-G), indicating that induced transcription of OBSCN-AS1 lncRNA promotes OBSCN transcriptional activation via chromatin remodeling and increased accessibility of the promoter for RNA polymerase II binding.

To further interrogate the regulation of OBSCN expression by OBSCN-AS1 lncRNA, we performed CRISPR-interference (CRISPRi; dCas9-KRAB) mediated knockdown of OBSCN-AS1 in MCF10A breast epithelial cells using three single guide RNAs, sgAS38, sgAS71, and sgAS74 targeting 38, 71 and 74 bp downstream of the OBSCN-AS1 TSS, respectively. CRISPRi targeting of OBSCN-AS1 resulted in robust downregulation of the transcript levels of both OBSCN-AS1 lncRNA variant 1 and 2 (FIG. 4H). Knockdown of OBSCN-AS1 resulted in a drastic reduction of OBSCN mRNA levels, too (FIG. 4I), further corroborating our findings with OBSCN-AS1 ASO treatment of sgAS2 transduced MDA-MB-231 (FIG. 4B-C) and MCF10A (FIG. 4D-E) cells. More importantly, ChIP-qPCR analysis revealed a significant decrease in RpbI occupancy and H3K4me3 levels alongside a marked enrichment in H3 lysine 9 trimethylation (H3K9me3), a repressive histone modification, not only at the OBSCN-AS1 promoter, as expected, but also at the OBSCN promoter in sgAS38 transduced MCF10A cells (FIG. 4J-L). Thus, OBSCN-AS1 knockdown in MCF10A breast epithelial cells modulates the epigenetic landscape (i.e., reduction of active and enrichment of repressive histone modifications) and decreases RNA polymerase II occupancy at the OBSCN promoter leading to suppressed OBSCN transcript expression, highlighting the key role of OBSCN-AS1 lncRNA on OBSCN's transcriptional regulation.

Collectively, our findings demonstrate that gain or loss of OBSCN-AS1 lncRNA expression in TNBC or non-tumorigenic breast epithelial cells, respectively, leads to altered histone modifications and RNA polymerase II occupancy at the OBSCN promoter, thus modulating its transcriptional activation.

### OBSCN-AS1/OBSCN Upregulation Suppresses Breast Cancer Cell Migration and Invasion

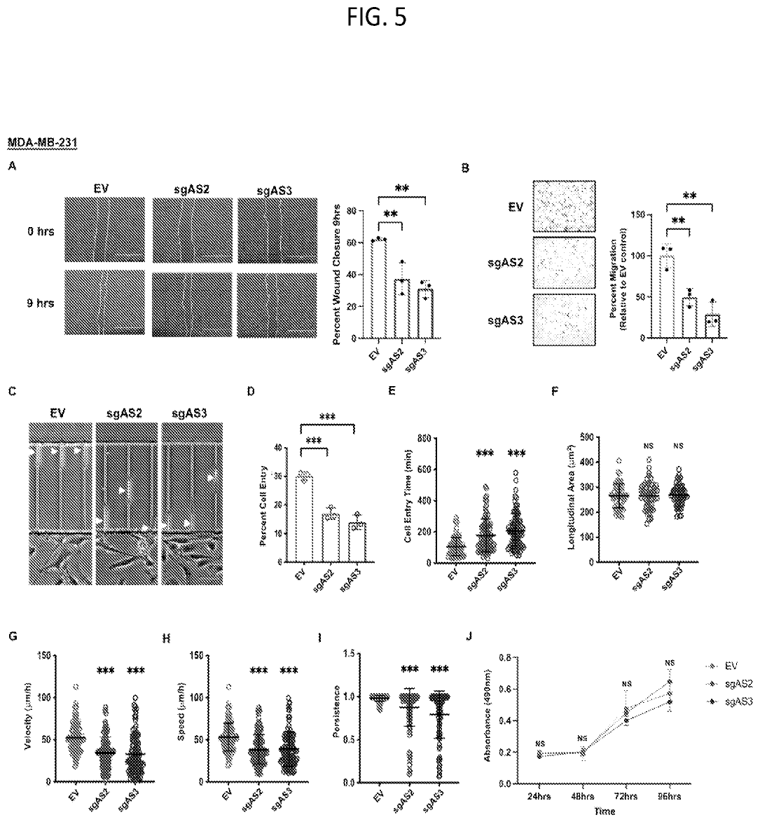

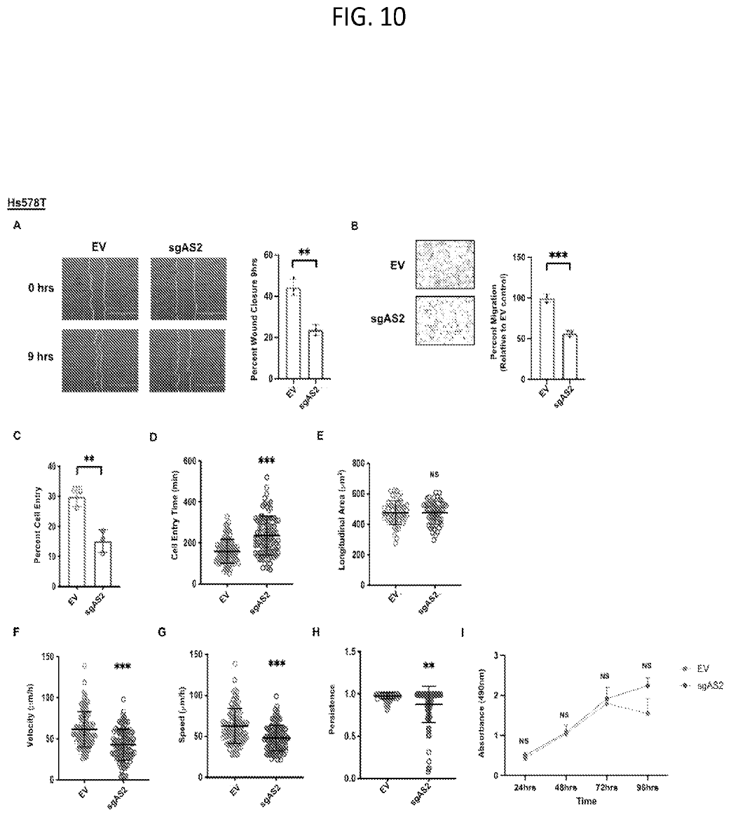

Earlier work from our group has demonstrated that the sole loss of OBSCN from non-tumorigenic breast epithelial cells transforms them to become highly migratory and invasive both in vitro and in vivo, two key phenotypes associated with cancer metastasis (Shriver et al., Oncogene 34, (2015), 4248-4259; Perry et al., Oncotarget 5, (2014), 8558-8568). We therefore interrogated the impact of the OBSCN-AS1/OBSCN upregulation in the largely aggressive and metastatic MDA-MB-231 and Hs578T TNBC cell lines by examining their migratory and invasive capabilities (Dai et al., J Cancer 8, (2017), 3131-3141; Holliday et al., Breast Cancer Res 13, (2011), 215; Neve et al., Cancer Cell 10, (2006), 515-527). Both MDA-MB-231 and Hs578T cells transduced with sgAS2 and/or sgAS3 exhibited dramatically reduced migration as assessed by wound healing (FIG. 5A and FIG. 10A) and transwell (FIG. 5B and FIG. 10B) assays. Given that cell migration is a critical step in the metastatic cascade where migratory cells degrade the surrounding extracellular matrix (ECM) to create their own path or move through pre-existing channel-like tracks, we proceeded to study single cell migration (Paul et al., Nat Rev Cancer 17, (2017), 131-140). To do so, we utilized a polydimethylsiloxane (PDMS)-based microfluidic device with confining narrow microchannels (10 mm in height×3 mm or×6 mm in width) (Wong et al., Cancer Res 79, (2019), 2878-2891 (2019); Yankaskas et al., Sci Adv 7, (2021); Zhao et al., Sci Adv 7, (2021)). Both MDA-MB-231 and Hs578T cells transduced with sgAS2 and/or sgAS3 exhibited markedly reduced ability to enter confining microchannels (FIG. 5C-D and FIG. 10C) and required significantly longer time to do so (FIG. 5E and FIG. 10D) compared to EV control cells. Importantly, the reduced ability of sgAS2 and/or sgAS3 transduced cells to enter narrow microchannels did not stem from changes in cell size (FIG. 5F and FIG. 10E). Moreover, OBSCN-AS1/OBSCN-expressing MDA-MB-231 and Hs578T cells migrated with considerably lower velocity (net displacement/time; FIG. 5G and FIG. 10F), speed (average displacement/time; FIG. 5H and FIG. 10G), and persistence (net displacement/total distance traveled; FIG. 5I and FIG. 10H) than control cells. Of note, in agreement with our prior work, there was no difference in cell proliferation between OBSCN-AS1/OBSCN-expressing MDA-MB-231 (FIG. 5J) and Hs578T (FIG. 10I) cells versus EV control cells (Perry et al., FASEB J 26, (2012) 2764-2775).

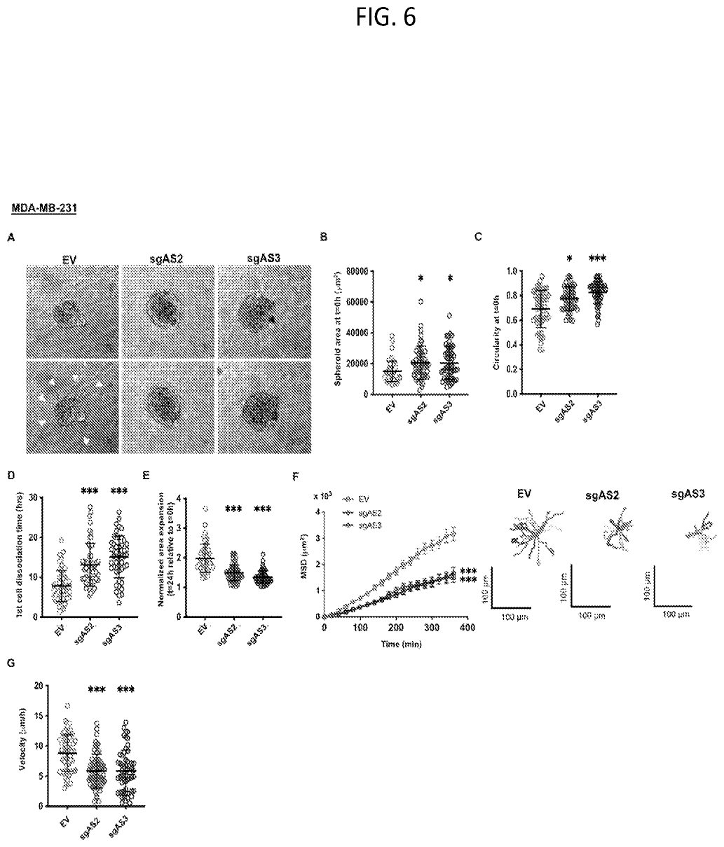

To extend the physiological relevance of our findings, we next examined the impact of the OBSCN-AS1/OBSCN upregulation in cell dissemination from 3-dimensional (3D) breast cancer spheroids embedded within 3D collagen gels (FIG. 6A). Despite the initial seeding of an equal number of cells across all groups, 3D spheroids generated from sgAS2 and sgAS3 expressing MDA-MB-231 cells displayed increased spheroid area relative to EV controls (FIG. 6B). This was due to the tendency of sgAS2 and sgAS3 transduced cells to coalesce and form aggregates, thereby giving rise to larger spheroids than EV control cells, which preferentially stayed as singlets at the time of initial seeding. Importantly, spheroids generated from sgAS2 and sgAS3 expressing cells exhibited markedly increased circularity relative to controls (FIG. 6C), which is indicative of their diminished cell dissemination. Consistent with this, sgAS2 and sgAS3 spheroids displayed significantly increased first cell dissociation time (FIG. 6D) and reduced area expansion (FIG. 6E) compared to controls. Moreover, in concert with our findings in confining channels, OBSCN-AS1/OBSCN expressing cells that disseminated from sgAS2 and sgAS3 spheroids displayed reduced migratory and invasive potentials in 3D collagen matrices relative to EV control cells, as evidenced by their lower mean square displacement (MSD; measure of the deviation of the position of a particle with respect to a reference position over time; FIG. 6F) and decreased velocity (FIG. 6G).

Taken together, these results demonstrate that restoration of OBSCN expression via OBSCN-AS1 activation in breast cancer cells significantly inhibits their dissemination from 3D spheroids as well as their migratory and invasive capacities, likely rendering them less metastatic.

### OBSCN Restoration Suppresses Breast Cancer Metastasis In Vivo

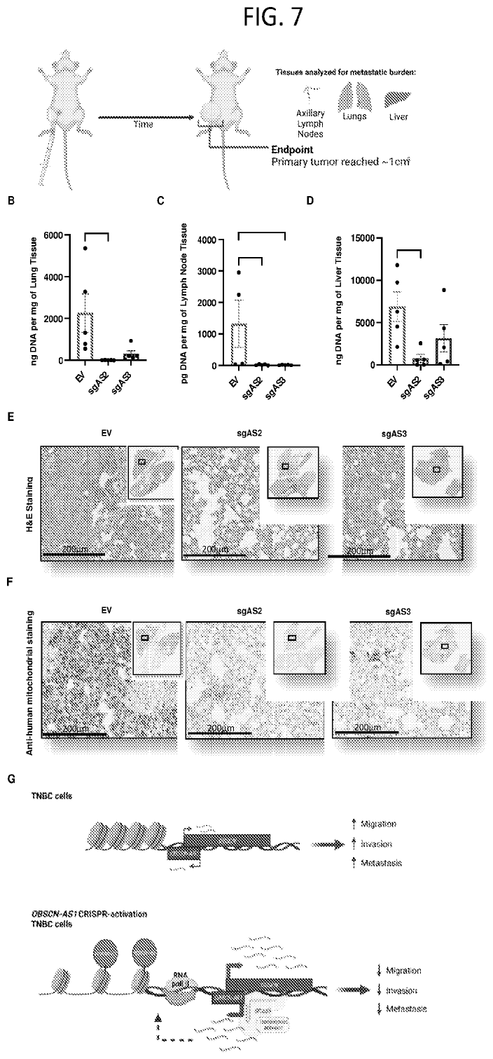

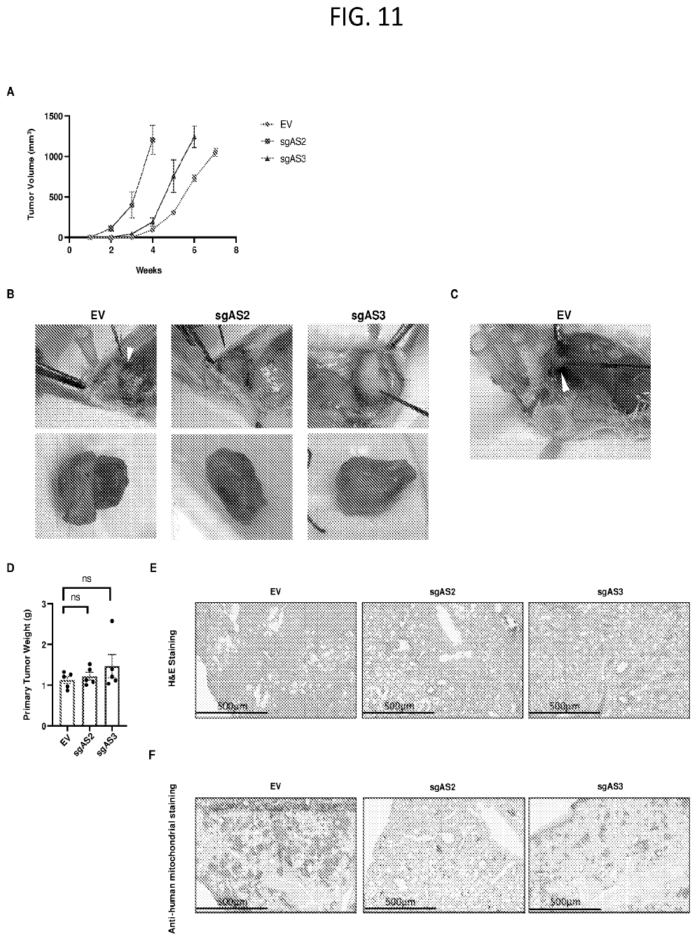

To examine whether the reduced dissemination from spheroids coupled with the lower migratory and invasive potentials of OBSCN-AS1/OBSCN-expressing MDA-MB-231 cells observed in vitro translate to alterations in their metastatic potential in vivo, we employed the well-established orthotopic breast cancer model. MDA-MB-231 cells transduced with EV control, sgAS2 or sgAS3 were inoculated into the 4^{th} mammary pad of female mice, with endpoint defined as the time that primary tumors reached ˜1 cm³ in volume (FIG. 7A). All groups developed primary tumors, however mice injected with MDA-MB-231 cells transduced with sgAS2 or sgAS3 reached terminal primary tumor volume earlier than mice injected with EV control cells (FIG. 11A). Interestingly, the primary tumors generated from sgAS2 and sgAS3 expressing cells were well-encapsulated, while tumors generated from EV control cells appeared to invade into the surrounding tissue and the peritoneum (FIG. 11B). Consistent with this, we observed visible macrometastases in the lymph nodes of at least one EV control animal (FIG. 11C). To follow-up on these intriguing observations, we measured metastatic tumor burden in distant organs by quantifying the amount of human DNA via qPCR using primers specific for the human long interspersed nuclear element (hLINE) gene. Of note, at the time of animal euthanasia and tissue harvesting, primary tumors from control and experimental groups were of similar weight (FIG. 11D), thus minimizing any potential confounding effect that primary tumor size may have on metastatic burden. Remarkably, our findings revealed significantly reduced (in most instances negligible) hLINE levels in the lungs (FIG. 7B), axillary lymph nodes (FIG. 7C) and liver (FIG. 7D) from animals injected with sgAS2 or sgAS3 transduced MDA-MB-231 cells compared to EV controls, indicating the presence of markedly decreased metastatic burden. Consistent with these findings, histochemical evaluation of lungs using hematoxylin and eosin (H&E) staining (FIG. 7E and FIG. 11E) showed a high abundance of human breast cancer cells throughout the parenchyma in the EV control, but not the sgAS2 or sgAS3 groups as confirmed by immunohistochemical detection of human mitochondria (FIG. 7F and FIG. 11F).

Taken together, our in vitro and in vivo findings demonstrate that OBSCN restoration in breast cancer cells drastically reduces metastatic burden, implicating OBSCN as a potent metastasis suppressor whose transcriptional regulation is under the control of the nuclear OBSCN-AS1 lncRNA (FIG. 7G).

### Materials and Methods

### Human Cell Lines and Cell Culture

MDA-MB-231, Hs578T, and HEK293T cells were purchased from ATCC and cultured in DMEM supplemented with 10% FBS and 1% penicillin-streptomycin. MCF10A cells were purchased from ATCC and cultured as described in (Perry et al., FASEB J 26, (2012) 2764-2775). Cells were maintained at 37° C. in a 5% CO₂ humidified tissue culture incubator and regularly checked for mycoplasma contamination via PCR using the MycoGuard Mycoplasma PCR Detection Kit (Genecopoeia, Rockville, MD).

### Generation of CRISPR-Activation (CRISPR-SAM) MDA-MB-231 and Hs578T Cells

Single guide RNAs (sgRNAs) targeting OBSCN (sgOBSCN1-4) were designed using the SAM Cas9 activator design tool and sgRNAs targeting OBSCN-AS1 (sgAS1-4) were designed using the Broad Institute CRISPick design tool. The sgRNA target sequences and oligonucleotide sequences are provided in Table 2. Cloning of Synergistic Activation Mediator (SAM) target sgRNAs was performed as in using the Golden-Gate sgRNA cloning protocol available from Addgene (Watertown, MA) (Konermann et al., Nature 517, (2015). 583-588; Joung et al., Nat Protoc 12, (2017), 828-863). Two lentiviral vectors were used: LentiMPHv2 (Addgene #89308) and LentiSAMv2 (Addgene #75112). Briefly, oligonucleotides were annealed and cloned into the LentiSAMv2 vector by golden gate reaction using BsmBI-v2 (NEB, Ipswich, MA). Plasmids were transformed into One-Shot Stbl3 chemically competent E. coli cells (Thermo Fisher, Waltham, MA) followed by plasmid DNA purification. Sequences were verified by Sanger sequencing (Genewiz, South Plainfield, NJ).

TABLE 2
CRISPR sgRNAs targeting OBSCN and OBSCN-ASI promoters

| | Target | Forward Oligo | Reverse Oligo |
|---|---|---|---|
| sgRNA Name | Sequence | (5′→3′) | (5′→3′) |
| sgOBSCN1 | GGTGGGGTGGGG | CACCGGGTGGGGTG | AAACGGGCCCGCCC |
| | GCGGGCCC (SEQ | GGGGCGGGCCC | CCACCCCACCC |
| | ID NO: 1) | (SEQ ID NO: 2) | (SEQ ID NO: 3) |
| sgOBSCN2 | CCGGGTCTCACCT | CACCGCCGGGTCTC | AAACCGGAGGGAG |
| | CCCTCCG (SEQ | ACCTCCCTCCG | GTGAGACCCGGC |
| | ID NO: 4) | (SEQ ID NO: 5) | (SEQ ID NO: 6) |
| sgOBSCN3 | GCGGCCGGAGTG | CACCGGCGGCCGGA | AAACGCAGCCCACA |
| | TGGGCTGC (SEQ | GTGTGGGCTGC | CTCCGGCCGCC |
| | ID NO: 7) | (SEQ ID NO: 8) | (SEQ ID NO: 9) |
| sgOBSCN4 | GCCGGCCTGGCC | CACCGGCCGGCCTG | AAACCCTGTGCCGG |
| | GGCACAGG (SEQ | GCCGGCACAGG | CCAGGCCGGCC |
| | ID NO: 10) | (SEQ ID NO: 11) | (SEQ ID NO: 12) |
| sgAS1 | AGGCACCGAGAA | CACCGAGGCACCGA | AAACCGGCTCCCTT |
| | GGGAGCCG (SEQ | GAAGGGAGCCG | CTCGGTGCCTC |
| | ID NO: 13) | (SEQ ID NO: 14) | (SEQ ID NO: 15) |
| sgAS2 | GGGAGGTAGCGG | CACCGGGGAGGTAG | AAACCCTTGCCCCC |
| | GGGCAAGG (SEQ | CGGGGGCAAGG | GCTACCTCCCC |
| | ID NO: 16) | (SEQ ID NO: 17) | (SEQ ID NO: 18) |
| sgAS3 | GGGGCAAGGAGG | CACCGGGGGCAAGG | AAACTCTCGGTGCC |
| | CACCGAGA (SEQ | AGGCACCGAGA | TCCTTGCCCCC |
| | ID NO: 19) | (SEQ ID NO: 20) | (SEQ ID NO: 21) |
| sgAS4 | CGGGGCACAGGG | CACCGCGGGGCACA | AAACCGAAGCCGCC |
| | CGGCTTCG (SEQ | GGGCGGCTTCG | CTGTGCCCCGC |
| | ID NO: 22) | (SEQ ID NO: 23) | (SEQ ID NO: 24) |
| sgCtrl | GTATTACTGATAT | CACCGGTATTACTG | AAACCCCACCAATA |
| | TGGTGGG (SEQ | ATATTGGTGGG | TCAGTAATACC |
| | ID NO: 25) | (SEQ ID NO: 26) | (SEQ ID NO: 27) |
| sgAS38 | GTCGGCCGAGAC | CACCGGTCGGCCGA | AAACTGCGCAATGT |
| | ATTGCGCA (SEQ | GACATTGCGCA | CTCGGCCGACC |
| | ID NO: 28) | (SEQ ID NO: 29) | (SEQ ID NO: 30) |
| sgAS71 | CGCCAAGTACGG | CACCGCGCCAAGTA | AAACCCTCGATGCC |
| | CATCGAGG (SEQ | CGGCATCGAGG | GTACTTGGCGC |
| | ID NO: 31) | (SEQ ID NO: 32) | (SEQ ID NO: 33) |
| sgAS74 | GAGCGCCAAGTA | CACCGGAGCGCCAA | AAACCGATGCCGTA |
| | CGGCATCG (SEQ | GTACGGCATCG | CTTGGCGCTCC |
| | ID NO: 34) | (SEQ ID NO: 35) | (SEQ ID NO: 36) |

For lentivirus production, HEK293T cells were cultured and ˜8.0×10⁶ cells were seeded in 15 cm culture dishes one day prior to transfection. Cells were polyethylenimine (PEI) transfected the next day at 50-70% confluency. For each transfection, 9 μg of plasmid containing the vector of interest, 3 μg of pMD2.G plasmid (Addgene #12259) and 12 μg of psPAX2 plasmid (Addgene #12260) were used with 72 μl of PEI. 18-24 h post-transfection the media was changed. Virus supernatant was harvested 48 h post-transfection, filtered through a 0.45 μm PVDF filter (MilliporeSigma, Burlington, MA), and lentivirus was concentrated using PEG Virus Precipitation Solution (Abcam, Cambridge, UK). Lentivirus was aliquoted and stored at −80° C. until ready for used. Lentivirus titers were determined using the qPCR Lentivirus Titer Kit (abm, Vancouver, Canada).

MDA-MB-231 and Hs578T cells were sequentially transduced with LentiMPH lentivirus and selected with hygromycin, and LentiSAM lentivirus and selected with blasticidin. Specifically, 3×10⁵ cells were seeded in 6-well plates with complete growth media one day prior to transduction. The next day the media was changed to 1 ml Opti-MEM supplemented with 8 μg/ml polybrene (AmericanBio, Canton, MA), and the appropriate volume of lentivirus was added to achieve a Multiplicity of Infection, MOI=1. 24 h post transduction, the media was removed and replaced with complete growth media. Selection agent was added 48 h post-transduction at the appropriate concentration, as determined by a kill curve: 1.2 mg/ml (MDA-MB-231) or 200 μg/ml (Hs578T) hygromycin, and 10 g/ml (MDA-MB-231) or 2 μg/ml (Hs578T) blasticidin. The duration of selection for each lentivirus was ˜7 days or until all cells in a control well (un-transduced cells) died from the selection.

### Generation of CRISPR-Interference (CRISPRi) MCF10A Cells

Single guide RNAs (sgRNAs) were designed using the Broad Institute CRISPick design tool to target near the OBSCN-AS1 transcription start site (TSS). The top 3 sgRNAs (sgAS38, sgAS71, and sgAS74) identified by CRISPick were used to target OBSCN-AS1 in MCF10A cells along with a non-targeting control sgRNA (sgCtrl). sgAS38, sgAS71 and sgAS74 target 38, 71 and 74 bp downstream of the OBSCN-AS1 TSS, respectively, in agreement with the stipulation that the optimal range for CRISPRi sgRNA design is −50 bp to +300 bp relative to the TSS. The sgRNA target sequences and oligonucleotide sequences are provided in Table 2. Given that OBSCN-AS1 and OBSCN share a genomic location (FIG. 1A), the identified guides theoretically target a region shared by the 2 genes. However, the selected guides target locations optimal for CRISPRi downregulation of OBSCN-AS1, but not OBSCN, as they are located >5 kb from the OBSCN TSS (i.e., sgAS38, sgAS71 and sgAS74 are located 5538, 5547, and 5550 bp, respectively, from the OBSCN TSS). Two lentiviral vectors were used: Lenti-dCas9-KRAB-blast (Addgene #89567) and LentiGuide-Puro (Addgene #52963). Briefly, oligonucleotides were annealed and cloned into the LentiGuide-Puro vector by golden date reaction using BsmBI-v2 (NEB, Ipswich, MA). Plasmids were transformed into One-Shot Stbl3 chemically competent E. coli cells (Thermo Fisher, Waltham, MA) followed by plasmid DNA purification. Sequences were verified by Sanger sequencing (Genewiz, South Plainfield, NJ). Lentivirus production was performed as described above using PEI transfection of HEK293T cells.

MCF10A cells were sequentially transduced with Lenti-dCas9-KRAB-blast lentivirus and selected with blasticidin, and LentiGuide-Puro lentivirus and selected with puromycin. Specifically, 3×10⁵ cells were seeded in 6-well plates with complete growth media one day prior to transduction. The next day, the media was changed to 1 ml Opti-MEM supplemented with 8 μg/ml polybrene (AmericanBio, Canton, MA), and the appropriate volume of lentivirus was added to achieve a Multiplicity of Infection, MOI=1. 24 h post-transduction, the media was removed and replaced with complete growth media. Selection agent was added 48 h post-transduction at the appropriate concentration, as determined by a kill curve: 6 μg/ml of blasticidin and 2 μg/ml of puromycin. The duration of selection for each lentivirus was ˜7 days or until all cells in a control well (un-transduced cells) died from the selection.

### RNA Extraction and RT-qPCR

Total RNA was extracted from cells using the Qiagen RNeasy Plus Mini Kit with gDNA eliminator columns (Qiagen, Germantown, MD), which selectively and efficiently remove genomic DNA. cDNA was synthesized from 2 g of total RNA using the SuperScript III First-Strand Synthesis System (Invitrogen, Thermo Fisher). KiCqStart Universal SYBR Green qPCR ReadyMix (Sigma, St. Louis, MO) was used for qPCR reactions. RNA expression levels were normalized to GAPDH using the ΔΔCt method. Three technical qPCR replicates for each of at least three biological replicates were performed; qPCR primer sequences are provided in Table 3.

TABLE 3
RT-qPCR primer sequences

| | Forward or | |
|---|---|---|
| Primer | Reverse | Primer sequence |
| Target | Primer | (5′→3′) |
| GAPDH | Forward | CTTTTGCGTCGCCAG (SEQ |
| | | ID NO: 37) |
| GAPDH | Reverse | TTGATGGCAACAATATCCAC |
| | | (SEQ ID NO: 38) |
| OBSCN | Forward | CAAGAGCCAGAAGTATGATG |
| | | (SEQ ID NO: 39) |
| OBSCN | Reverse | GTGAAGCAGTTTGCTTTTTC |
| | | (SEQ ID NO: 40) |
| OBSCN-AS1 | Forward | GTGTCCTCACAGCGTACCTC |
| variant 1 | | (SEQ ID NO: 41) |
| OBSCN-AS1 | Reverse | CCGTTACTCCAGAGACCCCT |
| variant 1 | | (SEQ ID NO: 42) |
| OBSCN-AS1 | Forward | GGTGAAACCCAAACATAGTC |
| variant 2 | | TCG (SEQ ID NO: 43) |
| OBSCN-AS1 | Reverse | TTGTGCAAGTGCAAGATTCT |
| variant 2 | | CA (SEQ ID NO: 44) |
| MALAT1 | Forward | CATTCGCTTAGTTGGTCTAC |
| | | (SEQ ID NO: 45) |
| MALAT1 | Reverse | TTCTACCGTTTTTCAGCTTC |
| | | (SEQ ID NO: 46) |

### RNA Fractionation and RT-qPCR

RNA fractionation from MCF10A cells was performed using PARIS (Protein and RNA Isolation System) kit (Thermo Fisher). Fractionated RNA samples were treated with DNase using the DNA-free DNA removal kit (Thermo Fisher) to remove trace genomic DNA contamination. 1 g DNase-treated, fractionated RNA was used for cDNA synthesis using the SuperScript III First-Strand Synthesis System (Invitrogen, Thermo Fisher). KiCqStart Universal SYBR Green qPCR ReadyMix (Sigma) was used for qPCR reactions. qPCR primer sequences are provided in Table 3. RT-qPCR data are presented as a percentage of the total amount of detected transcripts. Three technical qPCR replicates were performed for each of the three independent biological replicates.

### Dual-Luciferase Reporter Assay

Luciferase reporter assays were performed using the Nano-Glo Dual-Luciferase Reporter Assay System (Promega, Madison, WI). The promoter regions of OBSCN-AS1 and OBSCN were cloned from MCF10A genomic DNA and ligated into the NanoLuc luciferase pNL2.1[Nluc/Hygro] vector (Promega) at KpnI and XhoI sites (NEB), following PCR amplification with Amplitaq Gold 360 Master Mix (Thermo Fisher) and transformation in One Shot TOP10 chemically competent E. coli cells (Thermo Fisher). The authenticity of the obtained plasmids was verified by Sanger sequencing (Genewiz); primer sets are provided in Table 4.

TABLE 4
Luciferase assay cloning primer sequences;
restriction enzyme (KpnI and XhoI) sites
are underlined.

| | Forward or | |
|---|---|---|
| | Reverse | |
| Region | Primer | Primer sequence (5′→3′) |
| Region 1 | Forward | TAAGCAGGTACCACAGGACACT |
| | | CCTGTGCTCCCATG (SEQ ID |
| | | NO: 47) |
| Region 1 | Reverse | TGCTTACTCGAGCTTTAGTGGAG |
| | | CCACCCTCCAGG (SEQ ID |
| | | NO: 48) |
| Region 2 | Forward | TAAGCAGGTACCACTGCTAAGG |
| | | ACAAGGGTCTCTCG (SEQ ID |
| | | NO: 49) |
| Region 2 | Reverse | TGCTTACTCGAGCTTTAGTGGAG |
| | | CCACCCTCCAGG (SEQ ID |
| | | NO: 50) |
| Region 3 | Forward | TAAGCAGGTACCCCCTGGCCTC |
| | | CATCATTGCTAAGG (SEQ ID |
| | | NO: 51) |
| Region 3 | Reverse | TGCTTACTCGAGCTTTAGTGGAG |
| | | CCACCCTCCAGG (SEQ ID |
| | | NO: 52) |
| Region 4 | Forward | TAAGCAGGTACCACAGGACACT |
| | | CCTGTGCTCCCATG (SEQ ID |
| | | NO: 53) |
| Region 4 | Reverse | TGCTTACTCGAGCGAGAGACCC |
| | | TTGTCCTTAGCAGT (SEQ ID |
| | | NO: 54) |
| Region 5 | Forward | TAAGCAGGTACCACTGCTAAGG |
| | | ACAAGGGTCTCTCG (SEQ ID |
| | | NO: 55) |
| Region 5 | Reverse | TGCTTACTCGAGCCTTAGCAATG |
| | | ATGGAGGCCAGGG (SEQ ID |
| | | NO: 56) |
| OBSCN | Forward | TAAGCAGGTACCCCCTGGCCTC |
| promoter | | CATCATTGCTAAGG (SEQ ID |
| | | NO: 57) |
| OBSCN | Reverse | TGCTTACTCGAGCTTTAGTGGAG |
| promoter | | CCACCCTCCAGG (SEQ ID |
| | | NO: 58) |
| OBSCN-AS1 | Forward | TAAGCAGGTACCCAGTCCCAAC |
| promoter | | TCGTACCTCGCA (SEQ ID |
| | | NO: 59) |
| OBSCN-AS1 | Reverse | TGCTTACTCGAGCTCACCAGTCT |
| promoter | | TCTCTCCTGGCAG (SEQ ID |
| | | NO: 60) |

For transfection, 1×10⁴ MCF10A cells were seeded into the wells of a 96-well plate in triplicate one day prior to transfection using ViaFect Transfection Reagent (Promega). Cells were co-transfected (1:1 ratio) with the pNL2.1 constructs containing the OBSCN-AS1 or OBSCN promoter region segments and the transfection control firefly luciferase pGL4.50 [luc2/CMV/Hygro] vector, which is used to adjust for transfection efficiency differences. Positive control NanoLuc luciferase with TK promoter pNL1.1.TK [Nluc/TK] vector (Promega) and negative control empty pNL2.1. vector (Promega) were included in all experiments.

Luciferase activity was measured 48 h post-transfection according to the Nano-Glo Dual-Luciferase Reporter Assay System (Promega) protocol. Firefly luminescence and NanoLuc luminescence was measured using a FlexStation3 microplate reader (Molecular Devices, San Jose, CA). NanoLuc luciferase activities were normalized to firefly luciferase activities and expressed as relative light units (RLU). Three technical replicates were performed for each of the three independent biological replicates.

### Generation of Protein Lysates and Western Blotting

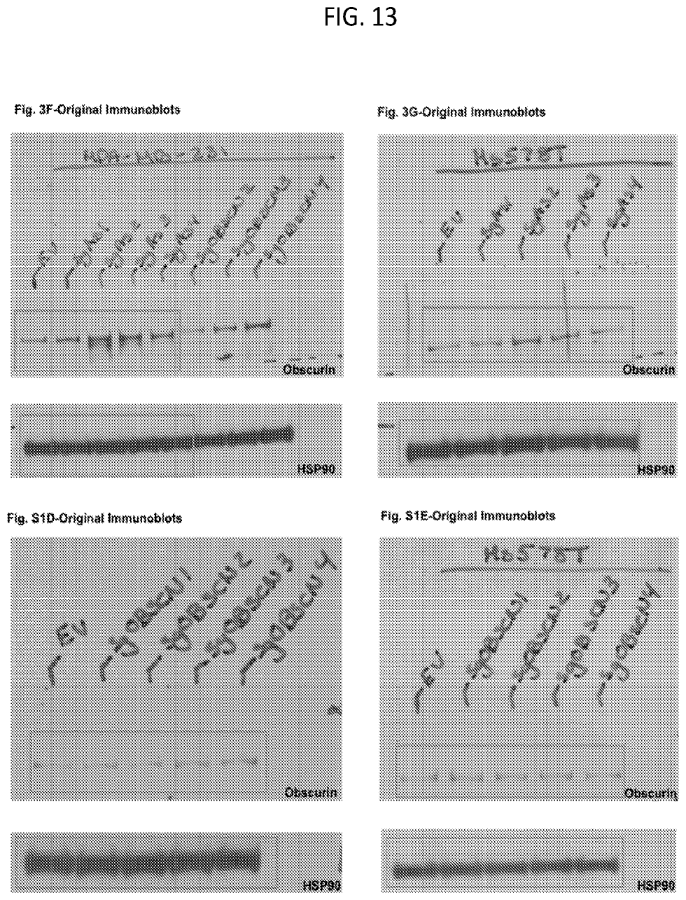

Cell lysates were prepared using radioimmunoprecipitation assay (RIPA, Sigma) buffer in the presence of Halt protease and phosphatase inhibitors (Thermo Fisher). Protein lysate concentration was determined using Quick Start Bradford Protein Assay (Bio-Rad, Hercules, CA), proteins were separated using NuPAGE 3-8% Tris-acetate SDS-PAGE gels and transferred onto nitrocellulose membranes for subsequent immunoblotting. Membranes were blocked for 2-3 h in 5% milk in Tris Buffered Saline (TBS) with 0.1% Tween-20 before overnight incubation at 4° C. with primary antibodies against Obscurin Ig58/59 rabbit polyclonal antibody at 0.5 g/ml and HSP90 (C45G5) Rabbit mAb (Cell Signaling Technology, #4877), followed by horseradish peroxidase (HRP) conjugated anti-rabbit secondary antibody (Cell Signaling Technology) (Shriver et al., Oncogene 34, (2015), 4248-4259). Immunoreactive bands were visualized with Pierce ECL Western Blotting Substrate (Thermo Fisher) or SignalFire ECL Reagent (Cell Signaling Technology, Danvers, MA) kits. Densitometric evaluation was performed with ImageJ (National Institute of Health, Bethesda, Maryland). At least three biological replicates were performed for each experiment. The original immunoblots are included in FIG. 13.

### Design and Transfection of Antisense Oligonucleotides (ASOs)

All ASOs used in this study were 2′-O-Methoxyethyl (2′-MOE) gapmers designed and obtained from Integrated DNA Technologies (IDT, Coralville, Iowa). MOE-gapmer ASOs are 20 nucleotides in length with a phosphorothioate backbone and the first and last 5 nucleotides are modified to include 2′O methoxyethoxy bases, which increase specificity and nuclease resistance. A total of 11 ASOs were designed and tested, and the 4 most potent ones were used for further experimentation. The sequences of the ASOs used in this study are provided in Table 5. 5×10⁵ MDA-MB-231 sgAS2 cells were seeded in a 6-well plate and transfected with individual non-targeting control (NC) or experimental ASOs at a 10 nM concentration using Lipofectamine 3000 Transfection Reagent (Invitrogen, Thermo Fisher). For combinatorial transfections, 5×10⁵ sgAS2 MDA-MB-231 and MCF10A cells were seeded and transfected with 20 nM of NC ASO or ASO-1 (10 nM) and ASO-4 (10 nM) for a total concentration of 20 nM. MCF10A cells were re-transfected 24 hr post-initial transfection to maximize transfection efficiency. Transfected cells were harvested within 48 h for RNA extraction and RT-qPCR analysis.

TABLE 5
OBSCN-ASI antisense oligonucleotide (ASO)
sequences and corresponding MOE-Gapmers

| ASO Name | ASO sequence | MOE-Gapmer |
|---|---|---|
| NC | GCGACTATACGCGCAATA | /52MOErG/*/i2MOErC/*/i2MOErG/*/i2MOErA/* |
| | TG (SEQ ID NO: 61) | /i2MOErC/*T*A*T*A*C*G*C*G*C*A*/i2MOErA/* |
| | | /i2MOErT/*/i2MOErA/*/i2MOErT/*/32MOErG/ |
| ASO-1 | TGTGAGAACACCCCCTTC | /52MOErT/*/i2MOErG/*/i2MOErT/*/i2MOErG/* |
| | AG (SEQ ID NO: 62) | /i2MOErA/*G*A*A*C*A*C*C*C*C*C*/i2MOErT/* |
| | | /i2MOErT/*/i2MOErC/*/i2MOErA/*/32MOErG/ |
| ASO-2 | CTCCACCTGTACCCCTTT | /52MOErC/*/i2MOErT/*/i2MOErC/*/i2MOErC/* |
| | GA (SEQ ID NO: 63) | /i2MOErA/*C*C*T*G*T*A*C*C*C*C*/i2MOErT/* |
| | | /i2MOErT/*/i2MOErT/*/i2MOErG/*/32MOErA/ |
| ASO-3 | GCTTCCCATGTATGCTTC | /52MOErG/*/i2MOErC/*/i2MOErT/*/i2MOErT/* |
| | AA (SEQ ID NO: 64) | /i2MOErC/*C*C*A*T*G*T*A*T*G*C*/i2MOErT/* |
| | | /i2MOErT/*/i2MOErC/*/i2MOErA/*/32MOErA/ |
| ASO-4 | TTTGGGTTTCACCAGTTC | /52MOErT/*/i2MOErT/*/i2MOErT/*/i2MOErG/* |
| | TT (SEQ ID NO: 65) | /i2MOErG/*G*T*T*T*C*A*C*C*A*G*/i2MOErT/* |
| | | /i2MOErT/*/i2MOErC/*/i2MOErT/*/32MOErT/ |

### Chromatin Immunoprecipitation-qPCR (ChIP-qPCR)

The SimpleChIP Enzymatic Chromatin IP Kit (Magnetic Beads) (Cell Signaling Technology, #9003) was used. Briefly, cells were fixed with formaldehyde and lysed, and chromatin was fragmented enzymatically with Micrococcal Nuclease in addition to brief pulse sonication. Adequate chromatin fragmentation was confirmed by the presence of DNA fragments approximately 150-900 bp (1 to 5 nucleosomes) in length on gel electrophoresis. 10 μg of digested, cross-linked chromatin was used per immunoprecipitation with ChIP-validated antibodies at 4° C. overnight with gentle rotation. The following ChIP-validated antibodies were used: positive control Histone H3 (D2B12) XP (Cell Signaling Technology, #4620), negative control normal Rabbit IgG (Cell Signaling Technology, #2729), Rbp1 NTD (D8L4Y) (Cell Signaling Technology, #14958), Tri-Methyl-Histone H3 (Lys4) (C42D8) (Cell Signaling Technology, #9751), and Tri-Methyl-Histone H3 (Lys9) (D4W1U) (Cell Signaling Technology, #13969). DNA was purified using DNA purification spin columns (Cell Signaling Technology) after reversal of protein-DNA cross-links by incubating chromatin samples with 5 M NaCl and Proteinase K at 65° C. for 2 h. Purified DNA was used as template for qPCR reactions using the SimpleChIP Universal qPCR Master Mix (Cell Signaling Technology, #88989); the ChIP-qPCR primers used are provided in Table 6. Signals obtained are expressed as percent (%) of the total input chromatin: Percent Input=2%×2^{(Ct 2% InputSample-Ct IP Sample)}. Three qPCR technical replicates were performed for each of the 3-4 independent biological replicates.

TABLE 6
ChIP-qPCR and hLINE qPCR primer sequences

| | Forward or | |
|---|---|---|
| | Reverse | Primer sequence |
| Primer | Primer | (5′→3′) |
| OBSCN- | Forward | CAAGTACGGCATCGAGGAGG |
| AS1_ChIP | | (SEQ ID NO: 66) |
| OBSCN- | Reverse | TCTGGCGTCTCGCAGATGTA |
| AS1_ChIP | | (SEQ ID NO: 67) |
| OBSCN_ChIP | Forward | TTGTCCCCGTCCAGTTTGAG |
| | | (SEQ ID NO: 68) |
| OBSCN_ChIP | Reverse | GGTCTCACCTCCCTCCGA |
| | | (SEQ ID NO: 69) |
| hLINE | Forward | TCACTCAAAGCCGCTCAACT |
| | | AC (SEQ ID NO: 70) |
| hLINE | Reverse | TCTGCCTTCATTTCGTTATG |
| | | TACC (SEQ ID NO: 71) |

### Wound Healing Assay

Transduced MDA-MB-231 and Hs578T cells were seeded in 6-well plates and cultured in complete growth medium for 24 h to reach confluency. Wound through the cell monolayer was generated using a 200 μl sterile pipette tip and cells were washed with PBS. Cells were cultured in complete growth medium, incubated at 37° C. 5% CO₂, and images were taken with the EVOS FL cell imaging system (Thermo Fisher) (4× objective) at time 0 h and 9 h. Cell migration was quantified as percentage (%) of wound closure using ImageJ from 3 independent experiments.

### Transwell Migration Assay

5×10⁴ transduced MDA-MB-231 or Hs578T cells in 500 μl of serum-free medium were seeded into the upper chamber of 24-well permeable inserts with 8.0 μm pore size (Corning, Corning, NY). The lower chamber of the wells was filled with complete growth medium containing 10% FBS. Chambers were incubated for 18 h (Hs578T cells) or 24 h (MDA-MB-231 cells) in a humidified tissue culture incubator at 37° C. 5% CO₂. Non-migrating cells were removed from the upper surface of the membrane with a cotton swab and migrated cells on the lower surface were stained with the Differential Quick Stain Kit (Polysciences, Inc., Warrington, PA) containing a fixative and two stain solutions (Modified Giemsa). Inserts were sequentially submerged in each solution for at least 5 min and then washed with distilled water. Membranes were carefully removed from the insert and placed on a slide for imaging. Migrated cells were quantified by counting at least 3 random fields from 3 independent experiments under an inverted light microscope (Olympus IX51, Center Valley, PA) (10× objective).

### Cell Proliferation Assay

The CellTiter 96 Aqueous One Solution Cell Proliferation Assay (Promega) was used according to the manufacturer's instructions. 5×10³ cells were seeded into 96-well plates with 100 μl of complete culture media. 20 μl of CellTiter 96 Aqueous One Solution reagent was added to each well and after 1 h incubation in a 37° C. 5% CO₂ humidified tissue culture incubator, absorbance was measured at 490 nm using a microplate reader; measurements were performed every 24 h up to 96 h post cell seeding. Three independent experiments were done for cell proliferation analysis.

### Microfluidic Device Fabrication, Cell Seeding, and Live-Cell Imaging and Analysis

PDMS-based microfluidic devices containing a series of parallel microchannels of prescribed height, width and length were fabricated as previously described (Yankaskas et al., Sci Adv 7, (2021); Zhao et al., Sci Adv 7, (2021). Specifically, for MDA-MB-231 cells microchannels of 10 μm in height, 3 μm in width and 200 μm in length were used, while for Hs578T cells microchannels of 10 μm in height, 6 μm in width and 400 μm in length were used. The microchannel dimensions were confirmed by a laser profilometer. Assembled microfluidic devices were incubated with rat tail collagen I (20 μg/ml, Thermo Fisher Scientific) for 1-2 h at 37° C. in the presence of 5% CO₂ prior to migration assays. Migration experiments were performed in DMEM containing 10% heat-inactivated FBS (Gibco) and 1% penicillin/streptomycin (10,000U/ml, Gibco). No chemotactic stimulus was applied in these experiments. 20 μl of cell suspension (4×10⁶ cells/ml) in serum-containing medium were added to the inlet well of the device. Prior to the migration experiments, medium was added to all inlet and outlet wells. Time-lapse images were recorded in 10 min intervals for up to 24 h in an inverted Nikon Eclipse Ti microscope (Nikon, Tokyo, Japan) equipped with a stage-top incubator (Okolab, Pozzuoli, Italy, or Tokai Hit, Shizuoka, Japan) at 37° C. and 5% CO₂, automated controls (NIS-Elements, Nikon) and a 10×/0.30 numerical aperture Ph1 objective. Cell migration analysis was performed as previously described (Mistriotis et al., J Cell Biol 218, (2019), 4093-4111; Zhao et al., Sci Adv 5, (2019), eaaw7243). Briefly, live videos were exported to ImageJ (National Institute of Health, Bethesda, Maryland). Cell entry time and percentage of cell entry were manually calculated from the videos obtained. Cell entry time was defined as the time interval from the time point that the leading edge of a cell-initiated entry into the microchannel until the entire cell had fully entered the microchannel. The tracks of individual cells that had fully entered the microchannels were obtained manually via Manual Tracking (Cordelieres F, Institut Curie, Orsay, France) plugin. Cell migration velocity was calculated using a custom MATLAB script (MathWorks, Natick MA).

### Spheroid Formation, and 3D Collagen Invasion Assay

Spheroids were formed as previously described (Dadakhujaev et al., Oncoscience 1, (2014), 229-240). Briefly, Geltrex™ LDEV-Free Reduced Growth Factor Basement Membrane Matrix (ThermoFisher) was diluted with DMEM containing 10% heat-inactivated FBS and 1% penicillin/streptomycin at 1:3 ratio. 50 μl of the diluted Geltrex™ were transferred to a 96-well plate (Falcon) and polymerized for 1 h at 37° C. and 5% CO₂ in a cell culture incubator. 2×10³ cells were suspended in 50 μl ice-cold Geltrex™ and gently plated in different wells pre-coated with polymerized Geltrex™ followed by incubation at 37° C. in a cell culture incubator. ˜1.5 h later, 150 μl of prewarmed DMEM containing 10% FBS and 1% penicillin/streptomycin was added in each well. The cell culture medium was replaced every two days, and spheroids were used in invasion assays 10-15 days later.

3D collagen invasion assays using spheroids were performed as previously described (Cheung et al., Cell 155, (2013), 1639-1651). Briefly, 3 ml of rat tail collagen type I (Corning) were mixed with 375 μl of 10×DMEM—low glucose (Sigma). The mixture pH was adjusted to physiological levels slowly with NaOH. 25 μl of the mixture were added to a 24 well-plate (Falcon) after 1 h incubation on ice, and then incubated at 37° C. in a cell culture incubator for 1 h. Spheroids were collected into 1.5 ml Eppendorf tubes by disrupting the Geltrex™ gently with ice cold DMEM. The Eppendorf tube was incubated in ice for >10 min to further depolymerize the Geltrex™. Spheroids were isolated by centrifugation (5,000 rpm) for 5 min and resuspended into 100 μl of the collagen mixture. Next, 100 μl of the spheroid-collagen mixture were plated in each well and incubated at 37° C. in a cell culture incubator for 1-1.5 h. Following collagen polymerization, 500 μl prewarmed cell culture media was added to each well. Time-lapse images were recorded in 20 min intervals for ˜35 h in an inverted Nikon Eclipse Ti microscope (Nikon) equipped with a stage-top incubator (Okolab or Tokai Hit) at 37° C. and 5% CO₂, automated controls (NIS-Elements, Nikon) and a 10×/0.30 numerical aperture Ph1 objective. First-cell dissociation times were obtained manually by measuring the time required for the first cell to fully detach from the spheroid using the NIS Element Software (Nikon). Cell velocity, cell trajectory, and mean squared displacement (MSD) were calculated using a custom-made MATLAB script. Circularity and normalized area expansion were measured using ImageJ by outlining the spheroids at t=0 and 24 h using polygonal regions of interest.

### Orthotopic Breast Cancer Model

Eight-to-twelve week old female NOD.Cg-Prkdc<scid>/J mice weighing 19-25 g were obtained from University of Maryland (Baltimore, MD) and fed food and water ad libitum. The mice were maintained in accordance with the Institutional Animal Care and Use Committee procedures and guidelines under an approved protocol. For injections, 2×10⁶ MDA-MB-231 Empty Vector (EV), sgAS2, or sgAS3 cells were suspended in 100 μL PBS and mixed with 50% of the total volume with Matrigel (Corning). Cell number was quantified via Countess® Automated Cell Counter (Thermo Fisher). The cell suspension for each line was then injected into the 4th mammary gland on the ventral surface of the abdomen of the female mice. Tumor volumes were measured by external caliper measurements weekly from the initial injection to the experimental endpoint. Tumors were measured along the two longest perpendicular axes in the x/y plane of each xenograft tumor to the nearest 0.1 mm with a digital caliper (Thomas Scientific, Inc.). Depth is assumed to be equivalent to the shortest of the perpendicular axes (y), and volume is calculated according to the: V=xy²/2 formula, as the standard practice for xenograft tumors. Signs of tumor ulceration or maximum tumor volume were recorded during each measurement as an experimental endpoint. Tissue samples were subsequently dissected for immunohistochemistry and hLINE qPCR analysis. Five animals (n=5) were used per group according to the minimum number of animals required to reach statistical significance following power analysis (n=DF/k+1; Sample Size Calculation in Animal Studies Using Resource Equation Approach; nih.gov).

hLINE qPCR Analysis of Metastatic Burden

DNA was extracted from frozen lung, axillary lymph node, and liver specimens using the DNeasy Blood and Tissue kit (Qiagen) according to the manufacturer's recommendations. Specifically, three (lung), one (lymph node), or five (liver) pieces (<25 mg per piece) were collected from each harvested organ, weighed, and recorded. Samples were lysed overnight at 56° C., and purification steps were followed as outlined in the manufacturer's protocol. DNA was eluted in 200 μl of DNA-RNA free water. Quantification of hLINE levels, which serve as proxy for the amount of human DNA present in mouse organs, was performed with qPCR as reported in with minor modifications (Tuntithavornwat et al., Cancer Lett 526, (2022), 155-167; Yankaskas et al., Nat Biomed Eng 3, (2019), 452-465). Briefly, qPCR was performed in a 20 μl reaction with the following components: 10 μl iTaq Universal SYBR Green Supermix (Bio-Rad), 1.5 μl of each 10 μM forward and reverse primers, 4.5 μl purified DNA and 2.5 μl water. With each qPCR experiment, serial dilutions of human DNA extracted from MDA-MB-231 cells using the DNeasy Blood and Tissue kit (Qiagen) were included to serve as standards. Results are shown as the amount of human DNA per mg of tissue-piece normalized to the corresponding primary tumor weight. Tissues from five animals per group were analyzed and three technical qPCR replicates were performed for each biological replicate; hLINE qPCR primer sequences are provided in Table 6.

### Immunohistochemistry and Pathology

Animals with primary tumor formation that reached the designated endpoint (i.e., tumors of 800 mm³-1000 mm³) were sacrificed. The lungs were removed, fixed in formalin for 24 h, embedded in paraffin wax, and serially sectioned (4 μm thick). All immunohistochemistry Human Mitochondria and H&E staining was performed by HistoWhiz (Brooklyn, NY).

### Strand-Specific RNA-Seq and Analysis

RNA extraction, library preparations, sequencing, and data analysis were conducted at Genewiz, LLC. (South Plainfield, NJ, USA) as follows. Extraction: Total RNA was extracted from frozen cell pellets using Qiagen RNeasy Plus Universal mini kit following the manufacturer's instructions (Qiagen, Hilden, Germany). Library Preparation with Stranded Poly A selection and HiSeq Sequencing: Extracted RNA samples were quantified using Qubit 2.0 Fluorometer (Life Technologies, Carlsbad, CA, USA) and RNA integrity was confirmed using Agilent TapeStation 4200 (Agilent Technologies, Palo Alto, CA, USA). RNA sequencing libraries were prepared using the NEBNext Ultra Directional RNA Library Prep Kit for Illumina according to the manufacturer's instructions (NEB, Ipswich, MA, USA). Briefly, mRNAs were first enriched with Oligo(dT) beads. Enriched mRNAs were fragmented for 15 min at 94° C., and first strand and second strand cDNAs were subsequently synthesized. cDNA fragments were end-repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified by using Qubit 2.0 Fluorometer (Invitrogen, Carlsbad, CA) and quantitative PCR (KAPA Biosystems, Wilmington, MA, USA). The sequencing libraries were pooled and clustered on 2 lanes of a flowcell. After clustering, the flowcell was loaded on the Illumina HiSeq instrument (4000 or equivalent) according to the manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification. Data Analysis: After investigating the quality of the raw data, sequence reads were trimmed to remove possible adapter sequences and nucleotides with poor quality using Trimmomatic v.0.36. The trimmed reads were mapped to the Homo Sapiens reference genome available on ENSEMBL using the STAR aligner v.2.5.2b. The STAR aligner is used as a splice aligner that detects splice junctions and incorporates them to help align the entire read sequences. BAM files were generated at this step. Unique gene hit counts were calculated by using feature Counts from the Subread package v.1.5.2. Only unique reads that fell within exon regions were counted. Since a strand-specific library preparation was performed, the reads were strand-specifically counted. After extraction of gene hit counts, the gene hit counts table was used for downstream differential expression analysis. Using DESeq2, a comparison of gene expression between the groups of samples was performed. The Wald test was used to generate p-values and Log 2 fold changes. Genes with p-values<0.01 and absolute log 2 fold changes >1 were called as differentially expressed for each comparison.

### TNBC Samples and Analysis

The GSE76250 dataset containing 165 TNBC and 33 normal samples was downloaded from the NCBI Gene Expression Omnibus database (GEO, https://www.ncbi.nlm.nih.gov/geo/) to evaluate the relationship between OBSCN and OBSCN-AS1 gene expression (Zhou et al., J Immunother Cancer 9, (2021). The 198 (165 TNBC and 33 normal controls) Affymetrix HTA-2_0 CEL files were downloaded, and data were extracted and Robust Multi-array Average (RMA) normalized using the Partek GS v6.6 platform (Partek Inc. St. Louis MO) (Bolstad et al., Bioinformatics 19, (2003), 185-193). Quality control using principle component and boxplot analysis detected two outlier samples, including one normal and one TNBC. Since their expression signal profiles differed from the others, suggesting technical differences, they were excluded from further analysis. The expression levels of OBSCN and OBSCN-AS1 in TNBC samples (n=164) were compared to normal controls (n=32) using two-tailed Student's t-test with statistical significance threshold set at p<0.05.

### Quantification and Statistical Analysis

Data are presented as mean±SD or SEM from at least 3 independent experiments. Data sets with Gaussian distributions were compared using two-tailed Student's t-test or one-way ANOVA followed by Tukey's, Dunnett's, or Fisher's LSD multiple comparisons test whenever appropriate. For non-Gaussian distributions, the nonparametric Mann-Whittney or Kruskal-Wallis test was used for comparisons between two or more groups, respectively. Two-way ANOVA followed by Dunn's or Sidak multiple comparisons test was used for comparisons between multiple groups with two independent variables. Statistical significance was defined as p<0.05. Calculations were performed using GraphPad Prism 7, 8 or 9 (GraphPad Software).

### Discussion

Earlier experimental work and bioinformatics analysis have documented the critical role of OBSCN in tumorigenesis (Guardia et al., Biochim Biophys Acta Rev Cancer 1876, (2021) 188567). Accordingly, OBSCN loss in normal breast epithelial cells has been causatively associated with anoikis escape, enhanced stemness, increased migration and invasion, and resistance to common chemotherapies (Shriver et al., Oncogene 34, (2015), 4248-4259; Perry et al., FASEB J 26, (2012) 2764-2775; Perry et al., Oncotarget 5, (2014), 8558-8568; Shriver et al., Oncotarget 7, (2016), 45414-45428). Consistent with these phenotypic alterations, obscurin levels are significantly decreased in advanced stage breast tumor biopsies, and breast cancer patients with low obscurin-expressing tumors display significantly reduced overall and relapse free survival, independently of their molecular subtype (Shriver et al., Oncogene 34, (2015), 4248-4259). Despite the compelling evidence implicating OBSCN loss in breast tumorigenesis and progression, its regulation has remained elusive, limiting any efforts to target and potentially restore its expression. Herein we found that the OBSCN transcriptional regulation is mediated by its non-coding gene partner, OBSCN-AS1 that encodes a nuclear lncRNA, through chromatin remodeling involving enrichment of H3K4me3 histone modification associated with an open chromatin conformation and recruitment of RNA polymerase II. In agreement with this, OBSCN and OBSCN-AS1 exhibit positively correlated expression (i.e., reduced levels) in breast cancer biopsies and cell lines. Remarkably, targeting of OBSCN-AS1 via CRISPR activation in highly aggressive TNBC cell lines restores OBSCN expression which in turn suppresses cell dissemination, migration, and invasion in vitro and metastasis in vivo, signifying the metastasis suppressor role of the OBSCN-AS1/OBSCN gene pair.

Considerable effort has been recently channeled towards the identification and roles of lncRNAs, which have been shown to regulate the expression of neighboring or distant genes by directly interacting with DNA, RNA, and proteins to modulate chromatin structure and function (Statello et al., Nat Rev Mol Cell Biol 22, 96-118 (2021)). These gene regulatory properties of nuclear lncRNAs may occur through diverse mechanisms, including RNA-mediated neutralization of positively charged histone tails, recruitment of chromatin modifiers, direct interaction with DNA and formation of R-loops, and epigenetic modifications (Dueva et al., Cell Chem Biol 26, (2019), 1436-1449 e1435; Wang et al., Nature 472, (2011), 120-124; Luo et al., Cancer Cell 36, (2019), 645-659 e648; Arab et al., Mol Cell 55, (2014), 604-614; Canzio et al. Cell 177, (2019), 639-653 e615). Conversely, the act of transcription or splicing of a lncRNA, rather than the lncRNA transcript per se, may impact chromatin structure by locally generating or alleviating steric hindrance and thus affecting the expression of neighboring genes (Statello et al., Nat Rev Mol Cell Biol 22, 96-118 (2021). Adding to the complexity of lncRNA-mediated gene regulation, these processes may occur independently or intertwine (Arab et al., Mol Cell 55, (2014), 604-614). Our findings demonstrating increased levels of H3K4me3 histone and RNA polymerase II at the OBSCN promoter following OBSCN-AS1 upregulation indicate that OBSCN-AS1 lncRNA most likely functions in a cis fashion (i.e., at its genomic locus) to induce OBSCN transcriptional activation.

OBSCN-AS1 lncRNA gives rise to two transcript variants with common and unique sequences, both of which preferentially localize to the nucleus. Considering that knockdown of both variants 1 and 2 via individual ASO-1 or combinatorial ASO-1 and ASO-4 treatment elicited the most robust downregulation of OBSCN transcript levels, we predict that both OBSCN-AS1 variants 1 and 2 coordinately regulate OBSCN activation, however future studies are warranted to address this question.

Remarkably, upregulation of OBSCN in TNBC cell lines via OBSCN-AS1 CRISPR activation resulted in significant reduction of their dissemination from 3D spheroids, migration, and invasion in vitro as well as metastasis in vivo. This is consistent with our prior work demonstrating that the sole loss of OBSCN in non-tumorigenic breast epithelial cells is sufficient to induce epithelial to mesenchymal transition (EMT) and cytoskeletal remodeling, and enhance cell morphodynamics and mechanosensitivity on soft surfaces (Shriver et al., Oncogene 34, (2015), 4248-4259; Stroka et al., Oncotarget 8, (2017), 54004-54020). Notably, recent experimental work has further implicated OBSCN loss in pancreatic cancer progression and metastasis, where loss of OBSCN in non-tumorigenic or moderately tumorigenic pancreatic epithelial cells results in faster cell migration via cytoskeletal reorganization involving reduced focal adhesion density, increased microtubule growth rate and faster actin dynamics, exacerbating primary tumor growth and metastasis (Tuntithavornwat et al., Cancer Lett 526, (2022), 155-167. Consistent with this, OBSCN levels are significantly reduced in pancreatic ductal adenocarcinoma tumor biopsies, similar to breast cancer biopsies (Tuntithavornwat et al., Cancer Lett 526, (2022), 155-167; Shriver et al., Oncogene 34, (2015), 4248-4259). Thus, it becomes apparent that OBSCN may have a pervasive suppressing role in cancer progression, which is in line with its nearly ubiquitous expression, albeit with varying abundance, among different tissues and organs (Guardia et al., Biochim Biophys Acta Rev Cancer 1876, (2021) 188567; Ackermann et al., PLoS One 9, (2014), e88162).

Intriguingly, although OBSCN upregulation via OBSCN-AS1 CRISPR activation in TNBC cells drastically reduces cell dissemination, confined migration, and invasion in vitro as well as metastasis in vivo, primary tumors showed an ostensibly faster growth rate. We postulate that this is due to the high propensity of OBSCN-AS1/OBSCN expressing cells to coalesce during the time of injection, which potentiates local tumor growth, in conjunction with their reduced ability to disperse. This is consistent with the behavior of the OBSCN-AS1/OBSCN transduced cells in 3D-spheroids and their unaltered proliferation rate. Importantly, our findings align with the notion that the relationship between primary tumor size and metastasis in breast cancer patients is not always linear (Sopik et al., Breast Cancer Res Treat 170, (2018), 647-656). Under the parallel model of breast cancer metastasis, primary tumor growth, lymph node metastasis, and distant metastasis are distinct processes governed by separate cell dynamics that occur simultaneously but independently (Sopik et al., Breast Cancer Res Treat 170, (2018), 647-656; Riggio et al., Br J Cancer 124, (2021). 13-26). Thus, the potential for metastasis is an inherent property of a distinct subpopulation of cells within the primary tumor (Riggio et al., Br J Cancer 124, (2021), 13-26). As such, early disseminated tumor cells are capable of escaping into the circulation and seeding local or distant metastases in parallel with, yet independently of, primary tumor growth (Riggio et al., Br J Cancer 124, (2021), 13-26). Such metastatic cells exhibit increased compliance and deformability, properties that allow them to withstand forces and readily navigate through confined spaces, in addition to enhanced collective migration, implicated as the predominant form of metastatic migration, rendering them highly aggressive (Lintz et al., J Biomech Eng 139, (2017)). Remarkably, our findings demonstrate that OBSCN-AS1/OBSCN restoration in TNBC cells is sufficient to drastically suppress these phenotypic alterations and inhibit metastasis.

Two main mechanisms have been suggested to underlie the frequent loss of tumor and metastasis suppressor genes, including increased mutational frequency and epigenetic dysregulation (Kazanets et al., Biochim Biophys Acta 1865, (2016), 275-288). Thus, an important point of consideration in our CRISPR activation approach is the restoration of a potentially mutant gene that could have deleterious effects. In silico evaluation of breast cancer databases revealed the presence of non-synonymous mutations in OBSCN in only 3.51% of queried invasive breast carcinoma samples, while their functional significance is unknown. Conversely, OBSCN was considerably hypermethylated in breast cancer samples exhibiting an average beta-value of >0.8 in a scale of 0-1 (Guardia et al., Biochim Biophys Acta Rev Cancer 1876, (2021) 188567; Rajendran et al., Oncotarget 8, (2017), 102263-102276). Notably, breast cancer biopsies displaying OBSCN hypermethylation contained significantly decreased OBSCN transcript levels compared to paired normal samples (Rajendran et al., Oncotarget 8, (2017), 102263-102276). In agreement with these observations, use of the Cosmic database revealed the presence of only two (c.2654-69C>T and c.21532+3030C>T) and one (c.9377C>T) missense heterozygous mutations (of unknown significance) in OBSCN in the TNBC MDA-MB-231 and Hs578T cell lines, respectively, used in our OBSCN-AS1/OBSCN CRISPR activation experiments. Yet, OBSCN transcript and proteins levels are significantly reduced in these cell lines as our studies indicated. We therefore postulate that OBSCN loss in breast cancer patients may be primarily driven by epigenetic modifications in the form of hypermethylation and/or aberrant downregulation of OBSCN-AS1, rather than increased OBSCN mutational frequency leading to unstable mRNA and/or protein.

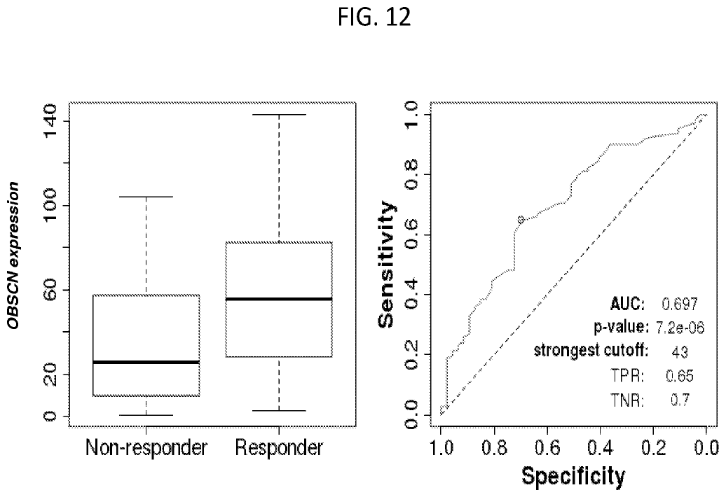

We posit that OBSCN is a novel metastasis suppressor in breast and likely other cancer types, whose transcriptional regulation is under the control of the OBSCN-AS1 lncRNA. Restoring OBSCN expression and functionality could therefore be of high pathophysiological significance as a novel, targeted, less toxic, and effective therapy for patients with obscurin-deficient tumors. This could be particularly beneficial for patients with obscurin-deficient triple negative breast tumors for whom current treatment options are limited to non-targeted chemotherapies and have the worst prognosis. Remarkably, using the ROC plotter tool, we found that higher OBSCN levels correlate with increased patient responsiveness to anthracyclines (i.e., doxorubicin and epirubicin), a non-targeted chemotherapy commonly used in breast cancer treatment. Consistent with this, OBSCN is a positive predictive biomarker for anthracycline therapy with an “Area Under the Curve” value of AUC=0.697 (FIG. 12). Moreover, CRISPR-Cas9 genome editing has recently emerged as a powerful tool for cancer therapy with applications in the discovery of novel target genes, dissection of chemical/genetic interactions and drug development, immunotherapeutic interventions, and gene editing in cell culture, preclinical models, and in some instances humans, prompting CRISPR clinical trials to slowly emerge. Given the highly selective action of OBSCN-AS1 on OBSCN as our findings demonstrate, restoring OBSCN expression via CRISPR activation may be highly efficacious with long-term translational potential as combination therapy (Cyranoski et al., Nature 539, (2016), 479; Martinez-Lage et al., Biomedicines 6, (2018); Lu et al., Proceedings: AACR Annual Meeting 2018; Cancer Research; Clinical Trials (2018)). This is particularly important in the case of OBSCN since the gigantic size of the obscurin transcript (20-24 kb) and protein (720-870 kDa) is prohibitive for adenoassociated viral (AAV) mediated gene delivery or peptide therapy, two approaches assessed in the clinic for restoring gene expression in cancer cells (Hacker et al., Cancers (Basel) 12 (2020); Marqus et al., J Biomed Sci 24, (2017), 21).

Collectively, our studies unravel novel aspects of the transcriptional regulation of OBSCN, a potent metastasis suppressor in breast cancer cells, via OBSCN-AS1 1ncRNA, implicating the OBSCN-AS1/OBSCN gene pair as a promising therapeutic target in metastatic breast cancer.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method for increasing OBSCN expression in a cell, comprising providing to the cell one or more agents that increases levels of OBSCN-AS1 lncRNA or a variant thereof in the cell.

2. The method of claim 1, wherein the cell is a cancer cell.

3. (canceled)

4. (canceled)

5. The method of claim 1, wherein the one or more agents comprises a nucleic acid encoding OBSCN-AS1 lncRNA or a variant thereof.

6. (canceled)

7. (canceled)

8. The method of claim 1, wherein the one or more agents binds to a promoter region of OBSCN-AS1 and increases expression of OBSCN-AS1 lncRNA in the cell, wherein the one or more agents comprises a CRISPR/Cas system comprising: (a) a nucleic acid encoding a sgRNA comprising a targeting domain which is complementary with a target sequence of the OBSCN-AS1 gene and (b) a nucleic acid encoding a Cas9 polypeptide or a variant thereof.

9. (canceled)

10. The method of claim 8, wherein the Cas9 polypeptide variant is nuclease deficient (dCas9), wherein the Cas9 polypeptide variant is fused to one or more polypeptide sequences capable of activating transcription and/or modifying histones.

11. (canceled)

12. The method of claim 8, wherein the target sequence is selected from the group consisting of:
a. SEQ ID NO:13;
b. SEQ ID NO:16;
c. SEQ ID NO:19; and
d. SEQ ID NO:22;

13. (canceled)

14. (canceled)

15. (canceled)

16. (canceled)

17. (canceled)

18. (canceled)

19. (canceled)

20. (canceled)

21. A method of treating cancer in a subject, comprising administering to the subject an effective amount of one or more agents that increases levels of OBSCN-AS1 lncRNA or a variant thereof in cancer cells of the subject.

22. (canceled)

23. (canceled)

24. The method of claim 21, wherein the one or more agents comprises a nucleic acid encoding OBSCN-AS1 lncRNA or a variant thereof.

25. The method of claim 21, wherein endogenous expression of OBSCN-AS1 lncRNA is increased by the one or more agents.

26. (canceled)

27. The method of claim 25, wherein the one or more agents binds to a promoter region of OBSCN-AS1 and increases expression of OBSCN-AS1 lncRNA in the cell, wherein the one or more agents comprises a CRISPR/Cas system comprising: (a) a nucleic acid encoding a sgRNA comprising a targeting domain which is complementary with a target sequence of the OBSCN-AS1 gene and (b) a nucleic acid encoding a Cas9 polypeptide or a variant thereof.

28. (canceled)

29. (canceled)

30. (canceled)

31. The method of claim 27, wherein the target sequence is selected from the group consisting of:
a. SEQ ID NO:13;
b. SEQ ID NO:16;
c. SEQ ID NO:19; and
d. SEQ ID NO:22;

32. (canceled)

33. (canceled)

34. (canceled)

35. (canceled)

36. (canceled)

37. (canceled)

38. (canceled)

39. The method of claim 21, wherein the treatment increases OBSCN expression and reduces cancer cell metastasis.

40. (canceled)

41. (canceled)

42. (canceled)

43. A CRISPR/Cas system for increasing OBSCN expression in a cell, comprising i) a nucleic acid encoding a sgRNA comprising a targeting domain which is complementary with a target sequence of the OBSCN-AS1 gene and ii) a nucleic acid encoding a Cas9 polypeptide or a variant thereof.

44. (canceled)

45. The system of claim 43, wherein the Cas9 polypeptide variant is nuclease deficient, wherein the Cas9 polypeptide variant is fused to one or more polypeptide sequences capable of activating transcription and/or modifying histones.

46. (canceled)

47. The system of claim 43, wherein the target sequence is selected from the group consisting of:
a. SEQ ID NO:13;
b. SEQ ID NO:16;
c. SEQ ID NO:19; and
d. SEQ ID NO:22;

48. (canceled)

49. (canceled)

50. (canceled)

51. (canceled)

52. (canceled)

53. (canceled)

54. (canceled)

55. A method of prognosing cancer in a subject, comprising
i) providing cancer cells from the subject;
ii) assaying the cells for expression of OBSCN and comparing OBSCN expression level to a control; and
iii) assaying the cells for expression of OBSCN-AS1 and comparing OBSCN-AS1 expression level to a control;
wherein reduced expression level of OBSCN and/or OBSCN-AS1 relative to the control indicate an increased probability for metastasis,
wherein normal or increased expression level of OBSCN and/or OBSCN-AS1 relative to the control indicate an increased sensitivity to an anthracycline chemotherapeutic agent.

56. The method of claim 55, wherein the subject is administered an effective amount of a therapeutic agent to treat cancer.

57. The method of claim 55, wherein the subject is administered an effective amount of an anthracycline chemotherapeutic agent.

58. (canceled)

59. The method of claim 55, wherein the cancer is breast cancer.

60. The method of claim 59, wherein the cancer is triple negative breast cancer.
